SEQUENCE LISTING (4 sequences)

SEQ ID NO: 1 - protein, 14 aa, Artificial Sequence
  Synthetically generated peptide
CCEYCCNPACTGCY

SEQ ID NO: 2 - protein, 14 aa, Artificial Sequence
  Synthetically generated peptide
CCEYCCDPACTGCY

SEQ ID NO: 3 - protein, 14 aa, Artificial Sequence
  Synthetically generated peptide
CCEYCCNPACTGCY

SEQ ID NO: 4 - protein, 14 aa, Artificial Sequence
  Synthetically generated peptide
CCEYCCNPACTGCY

(19) United States
(12) Patent Application Publication
(10) Pub. No.: US 2015/0031632 A1
(43) Pub. Date: Jan. 29, 2015

(54) Orally Disintegrating Compositions of Linaclotide

(71) Applicants: Forest Laboratories Holdings Limited, Hamilton (BM); Ironwood Pharmaceuticals, Inc., Cambridge, MA (US)

(72) Inventors: YUN MO, COMMACK, NY (US); MAHENDRA DEDHIYA, POMONA, NY (US); ANIL CHHETTRY, HOLTSVILLE, NY (US); ANGELIKA FRETZEN, SOMERVILLE, MA (US); STEVEN WITOWSKI, MELROSE, MA (US); ALFREDO GROSSI, SOMERVILLE, MA (US); HONG ZHAO, SOMERVILLE, MA (US)

(21) Appl. No.: 14/510,802

(22) Filed: Oct. 9, 2014

Related U.S. Application Data
(63) Continuation of application No. 13/389,919, filed on Sep. 21, 2012.
(60) Provisional application No. 61/233,314, filed on Aug. 12, 2009.

(51) Int. Cl. A61K 38/10; A61K 9/00; A61K 47/02; A61K 9/70; A61K 47/32; A61K 47/18; C07K 7/08; A61K 9/20

(52) U.S. Cl. CPC A61K 38/10; C07K 7/08; A61K 9/0056; A61K 9/2027; A61K 9/2013; A61K 9/2009; A61K 9/7007; A61K 47/32; A61K 47/183; A61K 47/02
USPC 514/21.1; 530/317

(57) ABSTRACT

The present invention relates to orally disintegrating or dissolving pharmaceutical compositions comprising linaclotide or pharmaceutically acceptable salts thereof, as well as to various methods and processes for the preparation and use of the compositions.

### CLAIM OF PRIORITY

This application is a continuation of, and claims priority under 35 U.S.C. §120 to U.S. patent application Ser. No. 13/389,919 filed on Feb. 10, 2012, which is the United States National Phase filing of PCT/US2010/045174 filed Aug. 11, 2010. This application also claims priority under 35 U.S.C. §119(e) to U.S. Provisional Application No. 61/233,314 filed on Aug. 12, 2009. The entire contents of the aforementioned applications are incorporated herein by reference.

### SEQUENCE LISTING

This application incorporates by reference in its entirety the Sequence Listing entitled “IW0079PCT1US1CON1_ST25”, which is 1.9 kilobytes in size and was last modified on Oct. 8, 2014, in computer readable-format (CRF) and electronic .txt format, filed electronically herewith.

### FIELD OF THE INVENTION

The present invention relates to stable orally disintegrating compositions, e.g., orally disintegrating tablets and orally disintegrating films, comprising linaclotide, and methods of treating conditions including irritable bowel syndrome and/or constipation, by administering the stable orally disintegrating compositions comprising linaclotide.

### BACKGROUND OF THE INVENTION

Various formulation techniques have been used to provide sustained and immediate release of pharmaceutically active agents, including orally disintegrating formulations of pharmaceutical agents. Typically, orally disintegrating formulations contain one or more disintegrating agents and optionally a film-forming agent and plasticizer. However, the specific components of these formulations depend greatly on the particular pharmaceutical agent and the desired formulation properties. For example, the formulation must be compatible with the pharmaceutical agent and also provide the necessary mechanical strength, taste-masking, dissolution performance, and stability properties.

Linaclotide is a peptide that is useful as an agonist of the guanylate cyclase C (GC-C) receptor in the treatment of gastrointestinal disorders. Linaclotide is described, for example, in U.S. Pat. Nos. 7,304,036 and 7,371,727, the contents of which are incorporated herein by reference in their entirety.

Tablet and capsule forms of linaclotide are disclosed in the '036 and '327 patents. However, tablets and capsules can be difficult for some patients to swallow, particularly for patients (e.g., elderly and pediatric patients) having gastrointestinal disorders. These difficulties associated with tablets and capsules can result in decreases in patient compliance.

Orally dissolving formulations of linaclotide are beneficial for many reasons. Their characteristic advantages such as capacity for administration without liquid lead to their suitability for treating patients having difficulty swallowing, such as children, the elderly and those with gastrointestinal disorders.

Despite the need for orally disintegrating compositions of linaclotide, difficulties exist in preparing such formulations due to the intrinsic and chemical instability of linaclotide (for example, induced by moisture-driven degradation reactions such as hydrolysis, deamidation, isomerization, and multimerization). These difficulties may be exacerbated when producing pediatric formulations having lower dosages of linaclotide, e.g., because the linaclotide is more dispersed and has greater surface area exposure to aqueous environments such as during preparation.

Accordingly, there is an existing and continual need for orally disintegrating formulations of linaclotide that provide reliable delivery of linaclotide, while also providing a dosing regimen that is straightforward and increases patient compliance.

### SUMMARY OF THE INVENTION

According to the present invention, it has now been found that linaclotide and its pharmaceutically acceptable salts can be formulated into stable orally disintegrating compositions. In addition, the present invention provides methods of treating conditions by administering the stable orally disintegrating compositions. The orally disintegrating formulations of the present invention may be used to treat various conditions, but is particularly suited to treat gastrointestinal disorders, such as irritable bowel syndrome (“IBS”) (for example, constipation-predominant IBS) and constipation (for example, chronic constipation).

According to some embodiments, methods of treating a gastrointestinal disorder comprising administering to a patient in need thereof, a therapeutically effective amount of a composition discussed herein is provided.

### DETAILED DESCRIPTION OF THE INVENTION

Orally disintegrating compositions, e.g., orally disintegrating tablets and orally disintegrating films, of linaclotide, as well as methods of treating gastrointestinal disorders, including irritable bowel syndrome (“IBS”) (for example, constipation-predominant IBS) and/or constipation (for example, chronic constipation) by administering the orally disintegrating compositions are provided herein.

Linaclotide is a peptide that consists of the amino acid sequence Cys₁ Cys₂ Glu₃ Tyr₄ Cys₅ Cys₆ Asn₇ Pro₈ Ala₉ Cys₁₀ Thr₁₁ Gly₁₂ Cys₁₃ Tyr₁₄. Any desired form of linaclotide may be used in the composition, for example, any pharmaceutically acceptable salt or hydrate of the peptide, any isolated and/or purified form thereof, or any disulfide form thereof. Disulfide forms of linaclotide are defined herein as linaclotide having one, two, or three of the following disulfide bonds between cysteinyl amino acids: a disulfide bond between Cys₁ and Cys₆, a disulfide bond between Cys₂ and Cys₁₀, and/or a disulfide bond between Cys₅ and Cys₁₃. For example, disulfide forms of linaclotide can comprise disulfide bonds between Cys₁ and Cys₆ and between Cys₂ and Cys₁₀. In addition, disulfide forms of linaclotide can comprise disulfide bonds between Cys₁ and Cys₆ and between Cys₅ and Cys₁₃. Moreover, disulfide forms of linaclotide can comprise disulfide bonds between Cys₂ and Cys₁₀ and between Cys₅ and Cys₁₃.

The orally disintegrating compositions may include any effective amount of linaclotide. In some embodiments, for example, the composition comprises from 0.05 μg to 6 mg of linaclotide. In some embodiments, for example, the composition comprises from 0.1 μg to 6 mg of linaclotide. In some embodiments, for example, the composition comprises from 25 μg to 6 mg of linaclotide. In some embodiments, the composition comprises from 25 μg to 2 mg of linaclotide, for example, from 50 μg to 1 mg of linaclotide. In some embodiments, for example, the composition comprises from 0.1 μg to 90 μg of linaclotide. In some embodiments, for example, the composition comprises from 0.1 μg to 45 μg of linaclotide. In some embodiments, for example, the composition comprises from 0.1 μg to 25 μg of linaclotide. In some embodiments, the composition comprises 0.05 μg, 0.1 μg, 0.15 μg, 0.25 μg, 0.5 μg, 0.75 μg, 1 μg, 1.5 μg, 2 μg, 2.5 μg, 3 μg, 3.5 μg, 4 μg, 4.5 μg, 5 μg, 7.5 μg, 10 μg, 15 μg, 20 μg, 25 μg, 30 μg, 35 μg, 40 μg, 45 μg, 50 μg, 60 μg, 75 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg or 1 mg of linaclotide. In some embodiments, the composition comprises from 100 μg to 600 μg of linaclotide. In some embodiments, the composition comprises 50 μg, 100 μg, 150 μg, 200 μg, 300 μg, 400 μg, 500 μg or 600 μg of linaclotide. In some embodiments, the composition comprises 75 μg, 150 μg, 300 μg, or 600 μg of linaclotide.

In some embodiments, the composition comprises 5 μg of linaclotide. In some embodiments, the composition comprises 7.5 μg of linaclotide. In some embodiments, the composition comprises 10 μg of linaclotide. In some embodiments, the composition comprises 20 μg of linaclotide. In some embodiments, the composition comprises 30 μg of linaclotide. In some embodiments, the composition comprises 40 μg of linaclotide. In some embodiments, the composition comprises 50 μg of linaclotide. In some embodiments, the composition comprises 60 μg of linaclotide. In some embodiments, the composition comprises 70 μg of linaclotide. In some embodiments, the composition comprises 80 μg of linaclotide. In some embodiments, the composition comprises 90 μg of linaclotide. In some embodiments, the composition comprises 100 μg of linaclotide. In some embodiments, the composition comprises 110 μg of linaclotide. In some embodiments, the composition comprises 120 μg of linaclotide. In some embodiments, the composition comprises 133 μg of linaclotide. In some embodiments, the composition comprises 150 μg of linaclotide. In some embodiments, the composition comprises 266 μg of linaclotide. In some embodiments, the composition comprises 300 μg of linaclotide. In some embodiments, the composition comprises 600 μg of linaclotide.

It has been found, in some embodiments, that the stability of orally disintegrating compositions of linaclotide can be increased or improved by including in the compositions a suitable amount of a sterically hindered primary amine (e.g., amino acid) component, a cation (e.g., metal cation) component, and/or a polymer component. These components increase or enhance the stability of orally disintegrating compositions of linaclotide, for example, by preventing, lessening, and/or decreasing degradation of linaclotide within the composition (for example, due to moisture-driven degradation reactions, e.g., hydrolysis, deamidation, and/or multimerization reactions). For instance, it has been found in some embodiments that addition or inclusion of a suitable amount of a cation (e.g., Mg²⁺, Ca²⁺, Zn²⁺) in the composition increases the stability of the composition against oxidative degradation of linaclotide. Moreover, it has been found in some embodiments that inclusion of a suitable amount of a sterically hindered primary amine (e.g., leucine) in the composition increases the stability of the composition against the formation of formaldehyde imine adducts of linaclotide, e.g., by sterically hindering linaclotide within the composition and/or by buffering the composition. Moreover, it has been found in some embodiments that inclusion of both a sterically hindered primary amine (e.g., leucine) and a cation (e.g., Ca²⁺) in suitable amounts in the composition increases the stability of the composition against the formation of hydrolysis products of linaclotide. It has also been found in some embodiments that inclusion of a suitable amount of a polymer (e.g., polyvinyl pyrrolidone or polyvinyl alcohol) in the orally disintegrating composition increases the stability of the composition for example by decreasing the mobility and/or reactivity of linaclotide within the composition, e.g., by forming a complex or matrix (for example, a glassy and/or rigid matrix) with linaclotide (e.g., by vitrification reaction), by preventing or lessening hydrogen bond formation between linaclotide and water molecules, and/or by enhancing the three-dimensional structural integrity of linaclotide. In this regard, it has been found in some embodiments that combining linaclotide in an orally disintegrating pharmaceutical composition with specific concentrations or molar ratios of the cation and sterically hindered primary amine causes a synergistic enhancement or improvement in the stability of linaclotide within the composition, for example as compared to similar compositions not containing the cation and/or sterically hindered primary amine and/or the same concentrations of these components.

The orally disintegrating composition can comprise any stabilizing amount of a sterically hindered primary amine component. For example, the composition can comprise a molar ratio of sterically hindered primary amine (e.g., amino acid) to linaclotide between 100:1 and 1:100. In some embodiments, the composition comprises a molar ratio of sterically hindered primary amine to linaclotide between 100:1 and 1:1. In some embodiments, the composition comprises a molar ratio of sterically hindered primary amine to linaclotide between 90:1 and 2:1. In some embodiments, the composition comprises a molar ratio of sterically hindered primary amine to linaclotide between 80:1 and 5:1. In some embodiments, the composition comprises a molar ratio of sterically hindered primary amine to linaclotide between 70:1 and 10:1. In some embodiments, the composition comprises a molar ratio of sterically hindered primary amine to linaclotide between 60:1 and 20:1. In some embodiments, the composition comprises a molar ratio of sterically hindered primary amine to linaclotide between 50:1 and 30:1. In some embodiments, the composition comprises a molar ratio of sterically hindered primary amine to linaclotide between 40:1 and 20:1. In some embodiments, the composition comprises a molar ratio of sterically hindered primary amine to linaclotide between 100:1 and 20:1. In some embodiments, the composition comprises a molar ratio of sterically hindered primary amine to linaclotide between 100:1 and 25:1. In some embodiments, the composition comprises a molar ratio of sterically hindered primary amine to linaclotide between 100:1 and 30:1. In some embodiments, the composition comprises a molar ratio of sterically hindered primary amine to linaclotide between 100:1 and 40:1. In some embodiments, the composition comprises a molar ratio of sterically hindered primary amine to linaclotide between 100:1 and 50:1. In some embodiments, the composition comprises a molar ratio of sterically hindered primary amine to linaclotide between 100:1 and 60:1. In some embodiments, the composition comprises a molar ratio of sterically hindered primary amine to linaclotide between 100:1 and 70:1. In some embodiments, the composition comprises a molar ratio of sterically hindered primary amine to linaclotide of at least 5:1. In some embodiments, the composition comprises a molar ratio of sterically hindered primary amine to linaclotide of at least 10:1. In some embodiments, the composition comprises a molar ratio of sterically hindered primary amine to linaclotide of at least 20:1. In some embodiments, the composition comprises a molar ratio of sterically hindered primary amine to linaclotide of at least 25:1. In some embodiments, the composition comprises a molar ratio of sterically hindered primary amine to linaclotide of at least 30:1. In some embodiments, the composition comprises a molar ratio of sterically hindered primary amine to linaclotide of at least 40:1.

Suitable sterically hindered primary amines for inclusion in the orally disintegrating composition are, for example, naturally-occurring amino acids (e.g., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine), synthetic amino acids (e.g., lanthionine, theanine or 1-amino cyclohexane), amino sugars (e.g., chitosane or glucosamine), or combination or mixtures thereof. In some embodiments, the composition comprises an amino acid selected from alanine, arginine, asparagine, aspartic acid, cysteine, glutamic acid, glutamine, histidine, isoleucine, leucine, lysine, methionine, phenylalanine, proline, serine, threonine, typtophan, tyrosine, valine, or a mixture thereof. In some embodiments, the composition comprises an amino acid selected from leucine, isoleucine, asparagine, glutamine, glutamic acid, histidine, cysteine, alanine, serine, threonine, tyrosine, proline, tryptophan, or a combination or mixture thereof. In some embodiments, the composition comprises an amino acid selected from leucine, isoleucine, methionine, alanine, or a combination or mixture thereof. In some embodiments, the composition comprises an amino acid selected from leucine, isoleucine, alanine, or a combination or mixture thereof. In some embodiments, the composition comprises an amino acid selected from leucine, isoleucine, methionine, or a combination or mixture thereof. In some embodiments, the composition comprises an amino acid selected from leucine, methionine, alanine, or a combination or mixture thereof. In some embodiments, the composition comprises leucine, methionine, or a mixture thereof. In some embodiments, the composition comprises leucine, isoleucine, or a mixture thereof. In some embodiments, the composition comprises leucine, alanine, or a mixture thereof. In some embodiments, the composition comprises leucine. In some embodiments, the composition comprises isoleucine. In some embodiments, the composition comprises methionine. In some embodiments, the composition comprises alanine.

The orally disintegrating composition can comprise any stabilizing amount of a cation (e.g., metal cation). For example, the composition can comprise a molar ratio of cation to linaclotide between 100:1 and 1:100. In some embodiments, the composition comprises a molar ratio of cation to linaclotide between 100:1 and 1:1. In some embodiments, the composition comprises a molar ratio of cation to linaclotide between 90:1 and 2:1. In some embodiments, the composition comprises a molar ratio of cation to linaclotide between 80:1 and 5:1. In some embodiments, the composition comprises a molar ratio of cation to linaclotide between 70:1 and 10:1. In some embodiments, the composition comprises a molar ratio of cation to linaclotide between 60:1 and 20:1. In some embodiments, the composition comprises a molar ratio of cation to linaclotide between 50:1 and 30:1. In some embodiments, the composition comprises a molar ratio of cation to linaclotide between 40:1 and 20:1. In some embodiments, the composition comprises a molar ratio of cation to linaclotide between 100:1 and 20:1. In some embodiments, the composition comprises a molar ratio of cation to linaclotide between 100:1 and 25:1. In some embodiments, the composition comprises a molar ratio of cation to linaclotide between 100:1 and 30:1. In some embodiments, the composition comprises a molar ratio of cation to linaclotide between 100:1 and 40:1. In some embodiments, the composition comprises a molar ratio of cation to linaclotide between 100:1 and 50:1. In some embodiments, the composition comprises a molar ratio of cation to linaclotide between 100:1 and 60:1. In some embodiments, the composition comprises a molar ratio of cation to linaclotide between 100:1 and 70:1. In some embodiments, the composition comprises a molar ratio of cation to linaclotide of at least 5:1. In some embodiments, the composition comprises a molar ratio of cation to linaclotide of at least 10:1. In some embodiments, the composition comprises a molar ratio of cation to linaclotide of at least 20:1. In some embodiments, the composition comprises a molar ratio of cation to linaclotide of at least 25:1. In some embodiments, the composition comprises a molar ratio of cation to linaclotide of at least 30:1. In some embodiments, the composition comprises a molar ratio of cation to linaclotide of at least 40:1. In some embodiments, the composition comprises a molar ratio of cation to linaclotide of at least 60:1.

Any suitable cation(s) can be included in the composition, for example, any suitable metal cation or organic cation. In some embodiments, the composition comprises a metal cation selected from calcium, potassium, magnesium, zinc, aluminum, iron, tin, manganese, chromium, cobalt, nickel, barium, sodium, or a combination or mixture thereof. In some embodiments, the composition comprises a metal cation selected from calcium, potassium, magnesium, zinc, aluminum, manganese, chromium, cobalt, nickel, barium, sodium, or a combination or mixture thereof. In some embodiments, the composition comprises a metal cation selected from aluminum, calcium, potassium, sodium, magnesium, manganese, zinc, or a combination or mixture thereof. In some embodiments, the composition comprises a metal cation selected from calcium, magnesium, manganese, zinc, or a combination or mixture thereof. In some embodiments, the composition comprises a divalent metal cation. In some embodiments, the composition comprises a divalent metal cation selected from Ca²⁺, Mg²⁺, Zn²⁺, Mn²⁺, or a combination or mixture thereof. In some embodiments, the composition comprises Mg²⁺. In some embodiments, the composition comprises Ca²⁺. In some embodiments, the composition comprises Zn²⁺. In some embodiments, the composition comprises aluminum. Moreover, the metal cation can be added to the composition in any suitable form, for example any pharmaceutically acceptable salt with any appropriate counterion. Suitable metal salts include, for example, calcium chloride, calcium carbonate, calcium acetate, magnesium chloride, magnesium acetate, zinc acetate, zinc chloride, or mixtures thereof. In some embodiments, the composition comprises calcium chloride, magnesium chloride, zinc acetate, or any combination or mixture thereof. In some embodiments, the composition comprises calcium chloride. In some embodiments, the composition comprises magnesium chloride. In some embodiments, the composition comprises zinc acetate. Suitable organic cations include, for example, ammonium hydroxide, D-arginine, L-arginine, t-butylamine, calcium acetate hydrate, calcium carbonate, calcium DL-malate, calcium hydroxide, choline, ethanolamine, ethylenediamine, glycine, L-histidine, L-lysine, magnesium hydroxide, N-methyl-D-glucamine, L-ornithine hydrochloride, potassium hydroxide, procaine hydrochloride, L-proline, pyridoxine, L-serine, sodium hydroxide, DL-triptophan, tromethamine, L-tyrosine, L-valine, carnitine, taurine, creatine malate, arginine alpha keto glutarate, ornithine alpha keto glutarate, spermine acetate, spermidine chloride, or combinations or mixtures thereof. In some embodiments, the organic cation is selected from the group consisting of N-methyl D-glucamine, choline, arginine, lysine, procaine, tromethamine (TRIS), spermine, N-methyl-morpholine, glucosamine, N,N-bis 2-hydroxyethyl glycine, diazabicycloundecene, creatine, arginine ethyl ester, amantadine, rimantadine, ornithine, taurine, citrulline, or a combination or mixture thereof.

The composition can contain any stabilizing amount of a polymer. In some embodiments, the composition is an orally disintegrating tablet and comprises between 0.1 and 75 wt. % of a polymer. In some embodiments, the composition is an orally disintegrating tablet and comprises between 0.1 and 55 wt. % of a polymer. In some embodiments, the composition is an orally disintegrating tablet and comprises between 0.1 and 35 wt. % of a polymer. In some embodiments, the composition is an orally disintegrating tablet and comprises between 0.1 and 30 wt. % of a polymer. In some embodiments, the composition is an orally disintegrating tablet and comprises between 0.1 and 25 wt. % of a polymer. In some embodiments, the composition is an orally disintegrating tablet and comprises between 1 and 25 wt. % of a polymer. In some embodiments, the composition is an orally disintegrating tablet and comprises between 5 and 25 wt. % of a polymer. In some embodiments, the composition is an orally disintegrating tablet and comprises between 10 and 25 wt. % of a polymer. In some embodiments, the composition is an orally disintegrating tablet and comprises between 15 and 25 wt. % of a polymer. In some embodiments, the composition is an orally disintegrating tablet and comprises between 0.1 and 22 wt. % of a polymer. In some embodiments, the composition is an orally disintegrating tablet and comprises between 1 and 22 wt. % of a polymer. In some embodiments, the composition is an orally disintegrating tablet and comprises between 5 and 22 wt. % of a polymer. In some embodiments, the composition is an orally disintegrating tablet and comprises between 10 and 22 wt. % of a polymer. In some embodiments, the composition is an orally disintegrating tablet and comprises between 0.1 and 20 wt. % of a polymer. In some embodiments, the composition is an orally disintegrating tablet and comprises between 1 and 20 wt. % of a polymer. In some embodiments, the composition is an orally disintegrating tablet and comprises between 5 and 20 wt. % of a polymer. In some embodiments, the composition is an orally disintegrating tablet and comprises between 10 and 20 wt. % of a polymer. In some embodiments, the composition is an orally disintegrating tablet and comprises between 0.01 and 15 wt. % of a polymer. In some embodiments, the composition is an orally disintegrating tablet and comprises between 0.01 and 10 wt. % of a polymer. In some embodiments, the composition is an orally disintegrating tablet and comprises between 0.01 and 5 wt. % of a polymer. In some embodiments, the composition is an orally disintegrating film and comprises between 0.1 and 95 wt. %, for example, between 5 and 95 wt. %, between 15 and 95 wt. %, between 25 and 95 wt. %, between 35 and 95 wt. %, between 45 and 95 wt. %, between 0.1 and 85 wt. %, between 1 and 85 wt. %, between 5 and 85 wt. %, between 15 and 85 wt. %, between 25 and 85 wt. %, between 35 and 85 wt. %, between 0.1 and 80 wt. %, between 1 and 80 wt. %, between 5 and 80 wt. %, between 15 and 80 wt. %, between 25 and 80 wt. %, between 35 and 80 wt. %, between 0.1 and 75 wt. %, between 1 and 75 wt. %, between 5 and 75 wt. %, between 15 and 75 wt. %, between 25 and 75 wt. %, between 35 and 75 wt. %, between 0.1 and 65 wt. %, between 1 and 65 wt. %, between 5 and 65 wt. %, between 15 and 65 wt. %, between 25 and 65 wt. %, between 35 and 65 wt. %, between 0.1 and 60 wt. %, between 1 and 60 wt. %, between 5 and 60 wt. %, between 15 and 60 wt. %, between 25 and 60 wt. %, or between 35 and 60 wt. % of a polymer. In some embodiments, the polymer acts as both a stabilizer and as a film forming agent within the orally disintegrating film. In some embodiments, the orally disintegrating composition comprises a molar ratio of polymer (e.g., PVP or PVA) to linaclotide between 80:1 and 300:1, for example, between 100:200:1, between 110:1 and 190:1, or even between 120:1 and 180:1. In some embodiments, the orally disintegrating composition comprises a molar ratio of polymer (e.g., PVP or PVA) to linaclotide greater than about 80:1, for example, greater than about 100:1, or even greater than about 120:1. In some embodiments, the orally disintegrating composition is an orally disintegrating tablet and comprises a weight ration of polymer (e.g., PVP or PVA) to linaclotide between 10:1 and 300:1, for example, between 80:1 and 200:1, between 100:1 and 180:1, or even between 110:1 and 150:1. In some embodiments, the orally disintegrating composition is an orally disintegrating film and comprises a weight ration of polymer (e.g., PVP or PVA) to linaclotide between 100:1 and 500:1, for example, between 200:1 and 400:1, between 250:1 and 350:1, or even between 300:1 and 350:1.

Suitable polymers for inclusion in the orally disintegrating compositions are, for example, polyvinyl pyrrolidone (PVP), polyvinyl alcohol (PVA), hydroxylpropyl methyl cellulose (HPMC), hydroxylpropyl cellulose (HPC), methyl cellulose, methacrylate polymers, cyclodextrin, dexrin, dextran, polyacrylic acid, chitosan, guar gum, xanthan gum, polyethylene oxide (e.g., polyethylene polypropylene oxide), poly(sodium vinylsulfonate), polyethylene glycol, poly(arginine), poly carbophil, polyvinyl pyrrolidone-co-vinyl acetate, a poloxamer (e.g., Pluronic® products available from BASF), alginate, trehalose, sucrose, inulin, or a combination or mixture thereof. In some embodiments, the composition comprises a polymer selected from PVP, PVA, methacrylate polymers, cyclodextrin, dextran, polyacrylic acid, chitosan, guar gum, xanthan gum, polyethylene oxide, polyethylene glycol, poly(arginine), poly carbophil, polyvinyl pyrrolidone-co-vinyl acetate, a poloxamer, or a combination or mixture thereof. In some embodiments, the composition comprises PVP, PVA, polyethylene oxide, or a mixture thereof. In some embodiments, the composition comprises PVP, PVA, or a mixture thereof. In some embodiments, the composition comprises PVP. In some embodiments, the composition comprises PVA.

In some embodiments, the orally disintegrating composition comprises two or more stabilizing agents. For example, the composition can include a stabilizing amount of a polymer and a stabilizing amount of a sterically hindered primary amine. Moreover, the composition can include a stabilizing amount of a polymer and a stabilizing amount of a cation (e.g., metal cation). In addition, the composition can include a stabilizing amount of a sterically hindered primary amine and a stabilizing amount of a cation (e.g., metal cation). In some embodiments, the composition comprises a stabilizing amount of a polymer, a stabilizing amount of a sterically hindered primary amine, and a stabilizing amount of a cation (e.g., metal cation).

In some embodiments, the orally disintegrating composition comprises a stabilizing amount of PVP and a stabilizing amount of an amino acid selected from alanine, arginine, asparagine, aspartic acid, cysteine, glutamic acid, glutamine, glycine, histidine, isoleucine, leucine, lysine, methionine, phenylalanine, proline, serine, threonine, typtophan, tyrosine, valine, or a mixture thereof. In some embodiments, the composition comprises a stabilizing amount of PVP and a stabilizing amount of an amino acid selected from alanine, arginine, asparagine, aspartic acid, cysteine, glutamic acid, glutamine, histidine, isoleucine, leucine, lysine, methionine, phenylalanine, proline, serine, threonine, typtophan, tyrosine, valine, or a mixture thereof. In some embodiments, the composition comprises a stabilizing amount of PVP and a stabilizing amount of leucine, isoleucine, methionine, alanine, or a combination or mixture thereof. In some embodiments, the composition comprises a stabilizing amount of PVP and a stabilizing amount of leucine.

In some embodiments, the orally disintegrating composition comprises a stabilizing amount of PVA and a stabilizing amount of an amino acid selected from alanine, arginine, asparagine, aspartic acid, cysteine, glutamic acid, glutamine, glycine, histidine, isoleucine, leucine, lysine, methionine, phenylalanine, proline, serine, threonine, typtophan, tyrosine, valine, or a mixture thereof. In some embodiments, the composition comprises a stabilizing amount of PVA and a stabilizing amount of an amino acid selected from alanine, arginine, asparagine, aspartic acid, cysteine, glutamic acid, glutamine, histidine, isoleucine, leucine, lysine, methionine, phenylalanine, proline, serine, threonine, typtophan, tyrosine, valine, or a mixture thereof. In some embodiments, the composition comprises a stabilizing amount of PVA and a stabilizing amount of leucine, isoleucine, methionine, alanine, or a combination or mixture thereof. In some embodiments, the composition comprises a stabilizing amount of PVA and a stabilizing amount of leucine.

In some embodiments, the orally disintegrating composition comprises a stabilizing amount of PVP and a stabilizing amount of a cation (e.g., metal cation). In some embodiments, the composition comprises a stabilizing amount of PVP and a stabilizing amount of a divalent metal cation. In some embodiments, the composition comprises a stabilizing amount of PVP and a stabilizing amount of Mg²⁺, Ca²⁺, Zn²⁺ or a salt thereof or a combination or mixture thereof. In some embodiments, the composition comprises a stabilizing amount of PVP and a stabilizing amount of Ca²⁺ or a salt thereof. In some embodiments, the composition comprises a stabilizing amount of PVP and a stabilizing amount of Mg²⁺ or a salt thereof. In some embodiments, the composition comprises a stabilizing amount of PVP and a stabilizing amount of Zn²⁺ or a salt thereof.

In some embodiments, the orally disintegrating composition comprises a stabilizing amount of PVA and a stabilizing amount of a cation (e.g., metal cation). In some embodiments, the composition comprises a stabilizing amount of PVA and a stabilizing amount of a divalent metal cation. In some embodiments, the composition comprises a stabilizing amount of PVA and a stabilizing amount of Mg²⁺, Ca²⁺, Zn²⁺ or a salt thereof or a combination or mixture thereof. In some embodiments, the composition comprises a stabilizing amount of PVA and a stabilizing amount of Ca²⁺ or a salt thereof. In some embodiments, the composition comprises a stabilizing amount of PVA and a stabilizing amount of Mg²⁺ or a salt thereof. In some embodiments, the composition comprises a stabilizing amount of PVA and a stabilizing amount of Zn²⁺ or a salt thereof.

In some embodiments, the orally disintegrating composition comprises a stabilizing amount of an amino acid selected from leucine, isoleucine, methionine, alanine; and a stabilizing amount of a divalent metal cation selected from Mg²⁺, Ca²⁺, Zn²⁺ or a salt thereof or a combination or mixture thereof. In some embodiments, the composition comprises a stabilizing amount of an amino acid selected from leucine, and isoleucine; and a stabilizing amount of a divalent metal cation selected from Mg²⁺, Ca²⁺ or a salt thereof or a combination or mixture thereof. In some embodiments, the composition comprises a stabilizing amount of an amino acid selected from leucine or methionine; and a stabilizing amount of a divalent metal cation selected from Ca²⁺, Zn²⁺ or a salt thereof or a combination or mixture thereof. In some embodiments, the composition comprises a stabilizing amount of leucine and a stabilizing amount of Ca²⁺ or a salt thereof. In some embodiments, the composition comprises a stabilizing amount of a cation and a stabilizing amount of a sterically hindered primary amine. In some embodiments, the composition comprises a cation and a sterically hindered primary amine in a molar ratio of cation:sterically hindered primary amine (e.g., Ca²⁺:leucine) of at least 1.5:1, e.g., at least 2:1, at least 2.5:1, at least 3:1, at least 4:1, or even at least 5:1 (for example, a molar ratio between 1.5:1 and 5:1, e.g., between 2:1 and 4:1).

In some embodiments, the orally disintegrating composition comprises (i) a stabilizing amount of PVP or PVA, (ii) a stabilizing amount of leucine, isoleucine, methionine, alanine, and (iii) a stabilizing amount of Mg²⁺, Ca²⁺, Zn²⁺ or a salt thereof or a combination or mixture thereof. In some embodiments, the composition comprises a stabilizing amount of PVP, a stabilizing amount of leucine, and a stabilizing amount of a metal cation. In some embodiments, the composition comprises a stabilizing amount of PVP, a stabilizing amount of leucine, and a stabilizing amount of Ca²⁺ or a salt thereof. In some embodiments, the composition comprises a stabilizing amount of PVP, a stabilizing amount of leucine, and a stabilizing amount of Mg²⁺ or a salt thereof. In some embodiments, the composition comprises a stabilizing amount of PVP, a stabilizing amount of leucine, and a stabilizing amount of Zn²⁺ or a salt thereof. In some embodiments, the composition comprises a stabilizing amount of PVA, a stabilizing amount of leucine, and a stabilizing amount of Ca²⁺ or a salt thereof. In some embodiments, the composition comprises a stabilizing amount of PVA, a stabilizing amount of leucine, and a stabilizing amount of Mg²⁺ or a salt thereof. In some embodiments, the composition comprises a stabilizing amount of PVA, a stabilizing amount of leucine, and a stabilizing amount of Zn²⁺ or a salt thereof.

In some embodiments, the composition is an orally disintegrating tablet and comprises (i) between 0.1 and 30 wt. % of a polymer, (ii) a sterically hindered primary amine (e.g., an amino acid) in a molar ratio of primary amine to linaclotide between 100:1 and 10:1, and (iii) a cation (e.g., a metal cation) in a molar ratio of cation to linaclotide between 100:1 and 40:1. In some embodiments, the composition is an orally disintegrating tablet and comprises (i) between 5 and 25 wt. % of a polymer, (ii) a sterically hindered primary amine (e.g., an amino acid) in a molar ratio of primary amine to linaclotide 100:1 and 30:1 (e.g., between 60:1 and 30:1 or even between 50:1 and 30:1), and (iii) a cation (e.g., a metal cation) in a molar ratio of cation to linaclotide between 100:1 and 60:1. In some embodiments, the composition is an orally disintegrating tablet and comprises (i) between 0.1 and 30 wt. % of a polymer selected from PVP and PVA, (ii) an amino acid selected from leucine, isoleucine, alanine, and methionine in a molar ratio of amino acid to linaclotide between 100:1 and 10:1, and (iii) a metal cation selected from Ca²⁺, Mg²⁺, and Zn²⁺ in a molar ratio of cation to linaclotide between 100:1 and 40:1. In some embodiments, the composition is an orally disintegrating tablet and comprises (i) between 5 and 25 wt. % of a polymer selected from PVP and PVA, (ii) an amino acid selected from leucine, isoleucine, alanine, and methionine in a molar ratio of amino acid to linaclotide 100:1 and 30:1 (e.g., between 60:1 and 30:1), and (iii) a metal cation selected from Ca²⁺, Mg²⁺, and Zn²⁺ in a molar ratio of cation to linaclotide between 100:1 and 60:1. In some embodiments, the composition is an orally disintegrating tablet and comprises (i) between 0.1 and 30 wt. % (e.g., between 5 and 25 wt. %) of PVP or PVA, (ii) leucine in a molar ratio of leucine to linaclotide between 100:1 and 30:1 (e.g., between 60:1 and 30:1 or even between 50:1 and 30:1), and (iii) Ca²⁺ in a molar ratio of Ca²⁺ to linaclotide between 100:1 and 60:1.

In some embodiments, the composition is an orally disintegrating film and comprises (i) between 45 and 99 wt. % of a polymer, (ii) a sterically hindered primary amine (e.g., an amino acid) in a molar ratio of primary amine to linaclotide between 100:1 and 10:1, and (iii) a cation (e.g., a metal cation) in a molar ratio of cation to linaclotide between 100:1 and 40:1. In some embodiments, the composition is an orally disintegrating film and comprises (i) between 45 and 70 wt. % of a polymer, (ii) a sterically hindered primary amine (e.g., an amino acid) in a molar ratio of primary amine to linaclotide 100:1 and 30:1 (e.g., between 60:1 and 30:1 or even between 50:1 and 30:1), and (iii) a cation (e.g., a metal cation) in a molar ratio of cation to linaclotide between 100:1 and 60:1. In some embodiments, the composition is an orally disintegrating film and comprises (i) between 45 and 99 wt. % of a polymer selected from PVP and PVA, (ii) an amino acid selected from leucine, isoleucine, alanine, and methionine in a molar ratio of amino acid to linaclotide between 100:1 and 10:1, and (iii) a metal cation selected from Ca²⁺, Mg²⁺, and Zn²⁺ in a molar ratio of cation to linaclotide between 100:1 and 40:1. In some embodiments, the composition is an orally disintegrating film and comprises (i) between 45 and 70 wt. % of a polymer selected from PVP and PVA, (ii) an amino acid selected from leucine, isoleucine, alanine, and methionine in a molar ratio of amino acid to linaclotide 100:1 and 30:1 (e.g., between 60:1 and 30:1), and (iii) a metal cation selected from Ca²⁺, Mg²⁺, and Zn²⁺ in a molar ratio of cation to linaclotide between 100:1 and 60:1. In some embodiments, the composition is an orally disintegrating film and comprises (i) between 45 and 99 wt. % (e.g., between 45 and 70 wt. %) of PVP or PVA, (ii) leucine in a molar ratio of leucine to linaclotide between 100:1 and 30:1 (e.g., between 60:1 and 30:1 or even between 50:1 and 30:1), and (iii) Ca²⁺ in a molar ratio of Ca²⁺ to linaclotide between 100:1 and 60:1. The orally disintegrating composition (e.g., orally disintegrating tablet) may also comprise any one or more filling agents. Suitable filling agents include, but are not limited to, starch, calcium carbonate, calcium sulfate, hydroxylpropylmethyl cellulose, fructose, methyl cellulose, dextrates, dextrose, dextran, lactitol, maltose, sucrose, sorbitol, isomalt, pregelatinized starch, dicalcium phosphate, microcrystalline cellulose, mannitol, gelatin, trehalose, erythitol, maltitol, lactose, glucose, or a combination thereof, or a mixture thereof. In some embodiments, the filling agent is isomalt. In some embodiments, the filling agent is gelatin. In some embodiments, the filling agent is mannitol. In some embodiments, the filling agent is pregelatinized starch. In some embodiments, the filling agent is microcrystalline cellulose.

The orally disintegrating composition (e.g., orally disintegrating tablet) can comprise any suitable concentration of filling agent. In some embodiments, for example, the composition comprises one or more filling agents in a concentration of 0.1-99% by weight, relative to the total weight of the composition. In some embodiments, for example, the composition comprises one or more filling agents in a concentration of 1-95 wt. % of filling agent(s), relative to the total weight of the composition. In some embodiments, for example, the composition comprises one or more filling agents in a concentration of 10-90 wt. % of filling agent(s), relative to the total weight of the composition. In some embodiments, for example, the composition comprises one or more filling agents in a concentration of 20-90 wt. % of filling agent(s), relative to the total weight of the composition. In some embodiments, for example, the composition comprises one or more filling agents in a concentration of 25-85 wt. % of filling agent(s), relative to the total weight of the composition. In some embodiments, for example, the composition comprises one or more filling agents in a concentration of 30-80 wt. % of filling agent(s), relative to the total weight of the composition. In some embodiments, for example, the composition comprises one or more filling agents in a concentration of 40-70 wt. % of filling agent(s), relative to the total weight of the composition. In some embodiments, for example, the composition comprises one or more filling agents in a concentration of 10-60 wt. % of filling agent(s), relative to the total weight of the composition. In some embodiments, for example, the composition comprises one or more filling agents in a concentration of 20-50 wt. % of filling agent(s), relative to the total weight of the composition. In some embodiments, the composition comprises one or more filling agents in a concentration of at least 20 wt. %, for example, at least 40 wt. %, at least 60 wt. %, at least 70 wt. %, at least 80 wt. %, or at least 90 wt. %, relative to the total weight of the composition.

In some embodiments, the orally disintegrating composition (e.g., orally disintegrating film) comprises one or more plasticizers. Suitable plasticizers include, but are not limited to, polyethylene glycol, propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol or combinations thereof. In exemplary embodiments, the concentration of the plasticizer in the formulation may be about 0 to about 30 wt %, for example, about 1 to about 20 wt %, about 0 to about 10 wt %, about 1 to about 5 wt %, or even 0 to about 4 wt %.

In some embodiments, the orally disintegrating composition (e.g., orally disintegrating film) comprises a film forming agent, a water-soluble polymer, a combination of two or more water-soluble polymers or a combination of a water-soluble polymer and a water-insoluble or -poorly-soluble polymer. Water soluble polymers that may be used in the orally dissolving formulations of the present invention include, but are not limited to, cellulose derivatives, synthetic polymers polyacrylates and natural gums. For example, the water soluble polymers used in the orally dissolving formulations of the present invention may include, but are not limited to, methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phtalate, cellulose acetate butyrate, amylose, dextran, casein, pullulan, gelatine, pectin, agar, carrageenan, xanthan gum, tragacanth, guar gum, acacia gum, arabic gum, polyethylene glycol, polyethylene oxide, polyvinyl pyrrolidone, polyvinyl alcohol, cyclodextrin, carboxyvinyl polymers, sodium alginate, polyacrylic acid, methylmethacrylate or mixtures thereof. In exemplary embodiments, the concentration of the water-soluble polymer in the formulation may be about 20% to about 90% (by weight), preferably between about 40% to about 80% (by weight).

One skilled in the art, with the benefit of this disclosure, will understand that other components may be included to enhance one or more properties of the orally disintegrating composition. In some embodiments, for example, the orally disintegrating compositions may include one or more disintegrants, lubricants, anti-caking additives, anti-microbial agents, antifoaming agents, emulsifiers, surfactants, buffering agents, and/or coloring agents.

Suitable disintegrants include, for example, agar-agar, calcium carbonate, microcrystalline cellulose, croscarmellose sodium, crospovidone, povidone, polacrilin potassium, sodium starch glycolate, potato or tapioca starch, other starches, pre-gelatinized starch, clays, other algins, other celluloses, gums, and mixtures thereof. In some embodiments, the disintegrant is crospovidone. In some embodiments, the disintegrant is croscarmellose sodium.

Suitable lubricants include, for example, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Evonik Degussa Co., Plano, Tex. USA), a pyrogenic silicon dioxide (CAB-O-SIL, Cabot Co., Boston, Mass. USA), and mixtures thereof.

Suitable anti-caking additives include, for example, calcium silicate, magnesium silicate, silicon dioxide, colloidal silicon dioxide, talc, and mixtures thereof.

Suitable anti-microbial additives that may be used, e.g., as a preservative for the linaclotide compositions, include, for example, benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, and mixtures thereof.

In some embodiments, the orally disintegrating compositions may comprise a taste-masking agent. Generally, any natural or synthetic flavoring agent or sweetening agent known in the art may be used in the orally dissolving formulations of the present invention. For example, suitable taste-masking agents include, but are not limited to, essential oils, water soluble extracts, sugar, monosaccharides, oligosaccharides, aldose, ketose, dextrose, maltose, lactose, glucose, fructose, sucrose, mannitol xylitol, D-sorbitol, erythritol, pentitol, hexitol, malitol, acesulfame potassium, talin, glycyrrhizin, sucralose, aspartame, saccharin, sodium saccharin, sodium cyclamate, eugenyl formate aldehyde flavorings and combinations thereof.

Exemplary aldehyde flavorings that may be used include, but are not limited to acetaldehyde (apple); benzaldehyde (cherry, almond); cinnamic aldehyde (cinnamon); citral, i.e., alpha citral (lemon, lime); neral, i.e., beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e., trans-2 (berry fruits); tolyl aldehyde (cherry, almond); veratraldehyde (vanilla); 2,6-dimethyl-5-heptenal, i.e., melonal (melon); 2-6-dimethyloctanal (green fruit); and 2-dodecenal (citrus, mandarin). In some embodiments, the taste-masking agents may include combination of acesulfame potassium and flavors. One skilled in the art with the benefit of the present disclosure will appreciate that other and further ingredients may be included in the orally dissolving formulations of the present invention. For example, a matrix-forming polymer permeation enhancer, substance for imparting mucoadhesive properties, or other auxiliary substances disclosed, for example, in U.S. Patent Publication No. 2005/0163830, the disclosure of which is hereby incorporated by reference in its entirety.

The composition may also comprise any suitable pharmaceutically acceptable carrier or medium. Suitable pharmaceutically acceptable carriers include, for example, any solvents, dispersants, pH buffering agents, coatings, absorption promoting agents, controlled release agents, and one or more inert excipients (e.g., filling agents, starches, polyols, granulating agents, microcrystalline cellulose, diluents, lubricants, binders, disintegrating agents), or the like. In addition, the compositions can contain any desired additional components, additives, and/or species, for example, surface active additives, dispersing additives, humectants, suspending agents, solubilizers, buffering agents, disintegrants, preservatives, colorants, flavorants, and the like. In some embodiments, the composition comprises one or more ion species that interact with linaclotide.

The composition can also comprise any suitable pH buffering agent. In some embodiments, the pH buffering agent is present in the composition in an amount sufficient to achieve the isoelectric point of linaclotide. In the regard, the composition can have any desired pH. In some embodiments, the composition has a pH of 2 to 5 (for example, a pH of 2 to 4.5, a pH of 2 4o 4, a pH of 2.5 to 4, a pH of 2.5 to 3.5, a pH of 2.5 to 3, or even a pH of 3).

In some embodiments, the composition comprises linaclotide and a hydrolysis product, e.g., a hydrolysis product comprising or having a structure of:

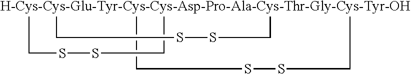

The composition can contain any desired concentration of the hydrolysis product. In some embodiments, the composition comprises less than 10 wt. % of the hydrolysis product. In some embodiments, the composition comprises less than 7 wt. % of the hydrolysis product. In some embodiments, the composition comprises less than 6 wt. % of the hydrolysis product. In some embodiments, the composition comprises less than 5 wt. % of the hydrolysis product. In some embodiments, the composition comprises less than 4 wt. % of the hydrolysis product. In some embodiments, the composition comprises less than 3 wt. % of the hydrolysis product. In some embodiments, the composition comprises less than 2 wt. % of the hydrolysis product. In some embodiments, the composition comprises less than 1 wt. % of the hydrolysis product. In some embodiments, the composition comprises between 0.01 and 10 wt. % of the hydrolysis product. In some embodiments, the composition comprises between 0.1 and 7 wt. % of the hydrolysis product. In some embodiments, the composition comprises between 0.1 and 5 wt. % of the hydrolysis product. In some embodiments, the composition comprises between 0.5 and 5 wt. % of the hydrolysis product. In some embodiments, the composition comprises between 1 and 5 wt. % of the hydrolysis product. In some embodiments, the composition comprises between 0.1 and 4 wt. % of the hydrolysis product. In some embodiments, the composition comprises between 0.5 and 4 wt. % of the hydrolysis product. In some embodiments, the composition comprises between 1 and 4 wt. % of the hydrolysis product. In some embodiments, the composition comprises between 0.1 and 3 wt. % of the hydrolysis product. In some embodiments, the composition comprises between 0.5 and 3 wt. % of the hydrolysis product. In some embodiments, the composition comprises between 1 and 3 wt. % of the hydrolysis product. In some embodiments, the composition comprises between 0.1 and 2.5 wt. % of the hydrolysis product. In some embodiments, the composition comprises between 0.5 and 2.5 wt. % of the hydrolysis product. In some embodiments, the composition comprises between 1 and 2.5 wt. % of the hydrolysis product. In some embodiments, the composition comprises between 0.1 and 2 wt. % of the hydrolysis product. In some embodiments, the composition comprises between 0.5 and 2 wt. % of the hydrolysis product. In some embodiments, the composition comprises between 1 and 2 wt. % of the hydrolysis product. In some embodiments, the composition comprises between 0.1 and 1.5 wt. % of the hydrolysis product. In some embodiments, the composition comprises between 0.5 and 1.5 wt. % of the hydrolysis product. In some embodiments, the composition comprises between 0.1 and 1 wt. % of the hydrolysis product. In some embodiments, the composition comprises between 0.5 and 1 wt. % of the hydrolysis product.

In some embodiments, the composition comprises linaclotide and a formaldehyde imine product, e.g., a formaldehyde imine product comprising or having a structure of:

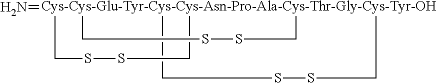

The composition can contain any desired concentration of the formaldehyde imine product. In some embodiments, the composition comprises less than 10 wt. % of the formaldehyde imine product. In some embodiments, the composition comprises less than 7 wt. % of the formaldehyde imine product. In some embodiments, the composition comprises less than 6 wt. % of the formaldehyde imine product. In some embodiments, the composition comprises less than 5 wt. % of the formaldehyde imine product. In some embodiments, the composition comprises less than 4 wt. % of the formaldehyde imine product. In some embodiments, the composition comprises less than 3 wt. % of the formaldehyde imine product. In some embodiments, the composition comprises less than 2 wt. % of the formaldehyde imine product. In some embodiments, the composition comprises less than 1 wt. % of the formaldehyde imine product. In some embodiments, the composition comprises between 0.01 and 10 wt. % of the formaldehyde imine product. In some embodiments, the composition comprises between 0.1 and 7 wt. % of the formaldehyde imine product. In some embodiments, the composition comprises between 0.1 and 5 wt. % of the formaldehyde imine product. In some embodiments, the composition comprises between 0.5 and 5 wt. % of the formaldehyde imine product. In some embodiments, the composition comprises between 1 and 5 wt. % of the formaldehyde imine product. In some embodiments, the composition comprises between 0.1 and 4 wt. % of the formaldehyde imine product. In some embodiments, the composition comprises between 0.5 and 4 wt. % of the formaldehyde imine product. In some embodiments, the composition comprises between 1 and 4 wt. % of the formaldehyde imine product. In some embodiments, the composition comprises between 0.1 and 3 wt. % of the formaldehyde imine product. In some embodiments, the composition comprises between 0.5 and 3 wt. % of the formaldehyde imine product. In some embodiments, the composition comprises between 1 and 3 wt. % of the formaldehyde imine product. In some embodiments, the composition comprises between 0.1 and 2.5 wt. % of the formaldehyde imine product. In some embodiments, the composition comprises between 0.5 and 2.5 wt. % of the formaldehyde imine product. In some embodiments, the composition comprises between 1 and 2.5 wt. % of the formaldehyde imine product. In some embodiments, the composition comprises between 0.1 and 2 wt. % of the formaldehyde imine product. In some embodiments, the composition comprises between 0.5 and 2 wt. % of the formaldehyde imine product. In some embodiments, the composition comprises between 1 and 2 wt. % of the formaldehyde imine product. In some embodiments, the composition comprises between 0.1 and 1.5 wt. % of the formaldehyde imine product. In some embodiments, the composition comprises between 0.5 and 1.5 wt. % of the formaldehyde imine product. In some embodiments, the composition comprises between 0.1 and 1 wt. % of the formaldehyde imine product. In some embodiments, the composition comprises between 0.5 and 1 wt. % of the formaldehyde imine product.

In some embodiments, the composition comprises linaclotide and an oxidation product, e.g., an oxidation product comprising or having a structure of:

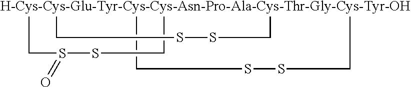

Alternatively, or in addition, the composition comprises linaclotide and an oxidation product having the depicted structure but wherein oxidation occurs at any one or more of the six depicted cysteinyl sulfurs. The composition can contain any desired concentration of the oxidation product. In some embodiments, the composition comprises less than 10 wt. % of the oxidation product. In some embodiments, the composition comprises less than 7 wt. % of the oxidation product. In some embodiments, the composition comprises less than 6 wt. % of the oxidation product. In some embodiments, the composition comprises less than 5 wt. % of the oxidation product. In some embodiments, the composition comprises less than 4 wt. % of the oxidation product. In some embodiments, the composition comprises less than 3 wt. % of the oxidation product. In some embodiments, the composition comprises less than 2 wt. % of the oxidation product. In some embodiments, the composition comprises less than 1 wt. % of the oxidation product. In some embodiments, the composition comprises between 0.01 and 10 wt. % of the oxidation product. In some embodiments, the composition comprises between 0.1 and 7 wt. % of the oxidation product. In some embodiments, the composition comprises between 0.1 and 5 wt. % of the oxidation product. In some embodiments, the composition comprises between 0.5 and 5 wt. % of the oxidation product. In some embodiments, the composition comprises between 1 and 5 wt. % of the oxidation product. In some embodiments, the composition comprises between 0.1 and 4 wt. % of the oxidation product. In some embodiments, the composition comprises between 0.5 and 4 wt. % of the oxidation product. In some embodiments, the composition comprises between 1 and 4 wt. % of the oxidation product. In some embodiments, the composition comprises between 0.1 and 3 wt. % of the oxidation product. In some embodiments, the composition comprises between 0.5 and 3 wt. % of the oxidation product. In some embodiments, the composition comprises between 1 and 3 wt. % of the oxidation product. In some embodiments, the composition comprises between 0.1 and 2.5 wt. % of the oxidation product. In some embodiments, the composition comprises between 0.5 and 2.5 wt. % of the oxidation product. In some embodiments, the composition comprises between 1 and 2.5 wt. % of the oxidation product. In some embodiments, the composition comprises between 0.1 and 2 wt. % of the oxidation product. In some embodiments, the composition comprises between 0.5 and 2 wt. % of the oxidation product. In some embodiments, the composition comprises between 1 and 2 wt. % of the oxidation product. In some embodiments, the composition comprises between 0.1 and 1.5 wt. % of the oxidation product. In some embodiments, the composition comprises between 0.5 and 1.5 wt. % of the oxidation product. In some embodiments, the composition comprises between 0.1 and 1 wt. % of the oxidation product. In some embodiments, the composition comprises between 0.5 and 1 wt. % of the oxidation product.

In some embodiments, the composition comprises linaclotide and an acetylation product, e.g., an acetylation product comprising or having:

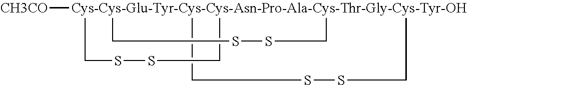

The composition can contain any desired concentration of the acetylation product. In some embodiments, the composition comprises less than 10 wt. % of the acetylation product. In some embodiments, the composition comprises less than 7 wt. % of the acetylation product. In some embodiments, the composition comprises less than 6 wt. % of the acetylation product. In some embodiments, the composition comprises less than 5 wt. % of the acetylation product. In some embodiments, the composition comprises less than 4 wt. % of the acetylation product. In some embodiments, the composition comprises less than 3 wt. % of the acetylation product. In some embodiments, the composition comprises less than 2 wt. % of the acetylation product. In some embodiments, the composition comprises less than 1 wt. % of the acetylation product. In some embodiments, the composition comprises between 0.01 and 10 wt. % of the acetylation product. In some embodiments, the composition comprises between 0.1 and 7 wt. % of the acetylation product. In some embodiments, the composition comprises between 0.1 and 5 wt. % of the acetylation product. In some embodiments, the composition comprises between 0.5 and 5 wt. % of the acetylation product. In some embodiments, the composition comprises between 1 and 5 wt. % of the acetylation product. In some embodiments, the composition comprises between 0.1 and 4 wt. % of the acetylation product. In some embodiments, the composition comprises between 0.5 and 4 wt. % of the acetylation product. In some embodiments, the composition comprises between 1 and 4 wt. % of the acetylation product. In some embodiments, the composition comprises between 0.1 and 3 wt. % of the acetylation product. In some embodiments, the composition comprises between 0.5 and 3 wt. % of the acetylation product. In some embodiments, the composition comprises between 1 and 3 wt. % of the acetylation product. In some embodiments, the composition comprises between 0.1 and 2.5 wt. % of the acetylation product. In some embodiments, the composition comprises between 0.5 and 2.5 wt. % of the acetylation product. In some embodiments, the composition comprises between 1 and 2.5 wt. % of the acetylation product. In some embodiments, the composition comprises between 0.1 and 2 wt. % of the acetylation product. In some embodiments, the composition comprises between 0.5 and 2 wt. % of the acetylation product. In some embodiments, the composition comprises between 1 and 2 wt. % of the acetylation product. In some embodiments, the composition comprises between 0.1 and 1.5 wt. % of the acetylation product. In some embodiments, the composition comprises between 0.5 and 1.5 wt. % of the acetylation product. In some embodiments, the composition comprises between 0.1 and 1 wt. % of the acetylation product. In some embodiments, the composition comprises between 0.5 and 1 wt. % of the acetylation product.

In some embodiments, the composition comprises linaclotide and any desired concentration of multimers. In some embodiments, the composition comprises less than 10 wt. % of multimer(s). In some embodiments, the composition comprises less than 7 wt. % of multimer(s). In some embodiments, the composition comprises less than 6 wt. % of multimer(s). In some embodiments, the composition comprises less than 5 wt. % of multimer(s). In some embodiments, the composition comprises less than 4 wt. % of multimer(s). In some embodiments, the composition comprises less than 3 wt. % of multimer(s). In some embodiments, the composition comprises less than 2 wt. % of multimer(s). In some embodiments, the composition comprises less than 1 wt. % of multimer(s). In some embodiments, the composition comprises between 0.01 and 10 wt. % of multimer(s). In some embodiments, the composition comprises between 0.1 and 7 wt. % of multimer(s). In some embodiments, the composition comprises between 0.1 and 5 wt. % of multimer(s). In some embodiments, the composition comprises between 0.5 and 5 wt. % of multimer(s). In some embodiments, the composition comprises between 1 and 5 wt. % of multimer(s). In some embodiments, the composition comprises between 0.1 and 4 wt. % of multimer(s). In some embodiments, the composition comprises between 0.5 and 4 wt. % of multimer(s). In some embodiments, the composition comprises between 1 and 4 wt. % of multimer(s). In some embodiments, the composition comprises between 0.1 and 3 wt. % of multimer(s). In some embodiments, the composition comprises between 0.5 and 3 wt. % of multimer(s). In some embodiments, the composition comprises between 1 and 3 wt. % of multimer(s). In some embodiments, the composition comprises between 0.1 and 2.5 wt. % of multimer(s). In some embodiments, the composition comprises between 0.5 and 2.5 wt. % of multimer(s). In some embodiments, the composition comprises between 1 and 2.5 wt. % of multimer(s). In some embodiments, the composition comprises between 0.1 and 2 wt. % of multimer(s). In some embodiments, the composition comprises between 0.5 and 2 wt. % of multimer(s). In some embodiments, the composition comprises between 1 and 2 wt. % of multimer(s). In some embodiments, the composition comprises between 0.1 and 1.5 wt. % of multimer(s). In some embodiments, the composition comprises between 0.5 and 1.5 wt. % of multimer(s). In some embodiments, the composition comprises between 0.1 and 1 wt. % of multimer(s). In some embodiments, the composition comprises between 0.5 and 1 wt. % of multimer(s).

In some embodiments, the composition comprises an effective amount of linaclotide and any desired amount of reduced form linaclotide. As used herein, the term “reduced form linaclotide” refers to linaclotide having no disulfide bonds between cysteine amino acids. In some embodiments, the composition comprises less than 10 wt. % of reduced form linaclotide. In some embodiments, the composition comprises less than 7 wt. % of reduced form linaclotide. In some embodiments, the composition comprises less than 6 wt. % of reduced form linaclotide. In some embodiments, the composition comprises less than 5 wt. % of reduced form linaclotide. In some embodiments, the composition comprises less than 4 wt. % of reduced form linaclotide. In some embodiments, the composition comprises less than 3 wt. % of reduced form linaclotide. In some embodiments, the composition comprises less than 2 wt. % of reduced form linaclotide. In some embodiments, the composition comprises less than 1 wt. % of reduced form linaclotide. In some embodiments, the composition comprises between 0.01 and 10 wt. % of reduced form linaclotide. In some embodiments, the composition comprises between 0.1 and 7 wt. % of reduced form linaclotide. In some embodiments, the composition comprises between 0.1 and 5 wt. % of reduced form linaclotide. In some embodiments, the composition comprises between 0.5 and 5 wt. % of reduced form linaclotide. In some embodiments, the composition comprises between 1 and 5 wt. % of reduced form linaclotide. In some embodiments, the composition comprises between 0.1 and 4 wt. % of reduced form linaclotide. In some embodiments, the composition comprises between 0.5 and 4 wt. % of reduced form linaclotide. In some embodiments, the composition comprises between 1 and 4 wt. % of reduced form linaclotide. In some embodiments, the composition comprises between 0.1 and 3 wt. % of reduced form linaclotide. In some embodiments, the composition comprises between 0.5 and 3 wt. % of reduced form linaclotide. In some embodiments, the composition comprises between 1 and 3 wt. % of reduced form linaclotide. In some embodiments, the composition comprises between 0.1 and 2.5 wt. % of reduced form linaclotide. In some embodiments, the composition comprises between 0.5 and 2.5 wt. % of reduced form linaclotide. In some embodiments, the composition comprises between 1 and 2.5 wt. % of reduced form linaclotide. In some embodiments, the composition comprises between 0.1 and 2 wt. % of reduced form linaclotide. In some embodiments, the composition comprises between 0.5 and 2 wt. % of reduced form linaclotide.

In some embodiments, the composition comprises between 1 and 2 wt. % of reduced form linaclotide. In some embodiments, the composition comprises between 0.1 and 1.5 wt. % of reduced form linaclotide. In some embodiments, the composition comprises between 0.5 and 1.5 wt. % of reduced form linaclotide. In some embodiments, the composition comprises between 0.1 and 1 wt. % of reduced form linaclotide. In some embodiments, the composition comprises between 0.5 and 1 wt. % of reduced form linaclotide.

In some embodiments, the composition comprises an effective amount of linaclotide and any desired amount of scrambled form linaclotide. As used herein, the term “scrambled form linaclotide” refers to linaclotide having disulfide bonds between Cys₁ and Cys₁₀, between Cys₁ and Cys₁₃, between Cys₁ and Cys₅, between Cys₁ and Cys₂, between Cys₂ and Cys₆, between Cys₂ and Cys₁₃, between Cys₂ and Cys₅, between Cys₅ and Cys₆, and/or between Cys₅ and Cys₁₀. In some embodiments, the composition comprises less than 10 wt. % of scrambled form linaclotide. In some embodiments, the composition comprises less than 7 wt. % of scrambled form linaclotide. In some embodiments, the composition comprises less than 6 wt. % of scrambled form linaclotide. In some embodiments, the composition comprises less than 5 wt. % of scrambled form linaclotide. In some embodiments, the composition comprises less than 4 wt. % of scrambled form linaclotide. In some embodiments, the composition comprises less than 3 wt. % of scrambled form linaclotide. In some embodiments, the composition comprises less than 2 wt. % of scrambled form linaclotide. In some embodiments, the composition comprises less than 1 wt. % of scrambled form linaclotide. In some embodiments, the composition comprises between 0.01 and 10 wt. % of scrambled form linaclotide. In some embodiments, the composition comprises between 0.1 and 7 wt. % of scrambled form linaclotide. In some embodiments, the composition comprises between 0.1 and 5 wt. % of scrambled form linaclotide. In some embodiments, the composition comprises between 0.5 and 5 wt. % of scrambled form linaclotide. In some embodiments, the composition comprises between 1 and 5 wt. % of scrambled form linaclotide. In some embodiments, the composition comprises between 0.1 and 4 wt. % of scrambled form linaclotide. In some embodiments, the composition comprises between 0.5 and 4 wt. % of scrambled form linaclotide. In some embodiments, the composition comprises between 1 and 4 wt. % of scrambled form linaclotide. In some embodiments, the composition comprises between 0.1 and 3 wt. % of scrambled form linaclotide. In some embodiments, the composition comprises between 0.5 and 3 wt. % of scrambled form linaclotide. In some embodiments, the composition comprises between 1 and 3 wt. % of scrambled form linaclotide. In some embodiments, the composition comprises between 0.1 and 2.5 wt. % of scrambled form linaclotide. In some embodiments, the composition comprises between 0.5 and 2.5 wt. % of scrambled form linaclotide. In some embodiments, the composition comprises between 1 and 2.5 wt. % of scrambled form linaclotide. In some embodiments, the composition comprises between 0.1 and 2 wt. % of scrambled form linaclotide. In some embodiments, the composition comprises between 0.5 and 2 wt. % of scrambled form linaclotide. In some embodiments, the composition comprises between 1 and 2 wt. % of scrambled form linaclotide. In some embodiments, the composition comprises between 0.1 and 1.5 wt. % of scrambled form linaclotide. In some embodiments, the composition comprises between 0.5 and 1.5 wt. % of scrambled form linaclotide. In some embodiments, the composition comprises between 0.1 and 1 wt. % of scrambled form linaclotide. In some embodiments, the composition comprises between 0.5 and 1 wt. % of scrambled form linaclotide.

In some embodiments, the composition comprises a total degradant concentration of less than about 10 wt. %. In some embodiments, the composition comprises a total degradant concentration of less than about 8 wt. %. In some embodiments, the composition comprises a total degradant concentration of less than about 7 wt. %. In some embodiments, the composition comprises a total degradant concentration of less than about 6.5 wt. %. In some embodiments, the composition comprises a total degradant concentration of less than about 6 wt. %. In some embodiments, the composition comprises a total degradant concentration of less than about 5.5 wt. %. In some embodiments, the composition comprises a total degradant concentration of less than about 5 wt. %. In some embodiments, the composition comprises a total degradant concentration of less than about 4 wt. %. In some embodiments, the composition comprises a total degradant concentration of less than about 3 wt. %. In some embodiments, the composition comprises a total degradant concentration of less than about 2.5 wt. %. In some embodiments, the composition comprises a total degradant concentration of less than about 2 wt. %. In some embodiments, the composition comprises a total degradant concentration of less than about 1 wt. %.

The composition, when administered, will dissolve to release linaclotide. The formulation may release the linaclotide over a period of time that is determined by a number of different factors. These factors include the dimensions of the formulation, the concentration of the linaclotide, and how the linaclotide is dispersed throughout the formulation. For example, by varying the thickness and surface area of the formulations the rate of dissolution may be adjusted. A thick formulation will dissolve more slowly than an otherwise similar thin formulation and may be desirable to administer high dosages of linaclotide.

In some embodiments, the orally disintegrating composition has a disintegration rate of less than about 30 seconds. In some embodiments, the orally disintegrating composition has a disintegration rate of less than about 25 seconds. In some embodiments, the orally disintegrating composition has a disintegration rate of less than about 20 seconds. In some embodiments, the orally disintegrating composition has a disintegration rate of less than about 15 seconds. In some embodiments, the orally disintegrating composition has a disintegration rate of less than about 10 seconds. In some embodiments, the orally disintegrating composition disintegrates in less than about 30 seconds after entering a use environment. In some embodiments, the orally disintegrating composition disintegrates in less than about 25 seconds after entering a use environment. In some embodiments, the orally disintegrating composition disintegrates in less than about 20 seconds after entering a use environment. In some embodiments, the orally disintegrating composition disintegrates in less than about 15 seconds after entering a use environment.

In some embodiments, the orally disintegrating composition releases at least about 75% of the linaclotide contained therein within 30 seconds of entering a use environment. In some embodiments, the orally disintegrating composition releases at least about 80% of the linaclotide contained therein within 30 seconds of entering a use environment. In some embodiments, the orally disintegrating composition releases at least about 85% of the linaclotide contained therein within 30 seconds of entering a use environment. In some embodiments, the orally disintegrating composition releases at least about 90% of the linaclotide contained therein within 30 seconds of entering a use environment. In some embodiments, the orally disintegrating composition releases at least about 95% of the linaclotide contained therein within 30 seconds of entering a use environment. In some embodiments, the orally disintegrating composition releases at least about 99% of the linaclotide contained therein within 30 seconds of entering a use environment.

In some embodiments, the orally disintegrating composition releases at least about 40% of the linaclotide contained therein within 15 seconds of entering a use environment. In some embodiments, the orally disintegrating composition releases at least about 50% of the linaclotide contained therein within 15 seconds of entering a use environment. In some embodiments, the orally disintegrating composition releases at least about 60% of the linaclotide contained therein within 15 seconds of entering a use environment. In some embodiments, the orally disintegrating composition releases at least about 70% of the linaclotide contained therein within 15 seconds of entering a use environment. In some embodiments, the orally disintegrating composition releases at least about 80% of the linaclotide contained therein within 15 seconds of entering a use environment. In some embodiments, the orally disintegrating composition releases at least about 85% of the linaclotide contained therein within 15 seconds of entering a use environment. In some embodiments, the orally disintegrating composition releases at least about 90% of the linaclotide contained therein within 15 seconds of entering a use environment. In some embodiments, the orally disintegrating composition releases at least about 95% of the linaclotide contained therein within 15 seconds of entering a use environment.

In some embodiments, the orally disintegrating composition releases at least about 75% of the linaclotide contained therein within 30 seconds of entering the oral cavity of a patient. In some embodiments, the orally disintegrating composition releases at least about 80% of the linaclotide contained therein within 30 seconds of entering the oral cavity of a patient. In some embodiments, the orally disintegrating composition releases at least about 85% of the linaclotide contained therein within 30 seconds of entering the oral cavity of a patient. In some embodiments, the orally disintegrating composition releases at least about 90% of the linaclotide contained therein within 30 seconds of entering the oral cavity of a patient. In some embodiments, the orally disintegrating composition releases at least about 95% of the linaclotide contained therein within 30 seconds of entering the oral cavity of a patient. In some embodiments, the orally disintegrating composition releases at least about 99% of the linaclotide contained therein within 30 seconds of entering the oral cavity of a patient.

In some embodiments, the orally disintegrating composition releases at least about 75% of the linaclotide contained therein within 30 seconds of contacting saliva having a pH greater than 5. In some embodiments, the orally disintegrating composition releases at least about 80% of the linaclotide contained therein within 30 seconds of contacting saliva having a pH greater than 5. In some embodiments, the orally disintegrating composition releases at least about 85% of the linaclotide contained therein within 30 seconds of contacting saliva having a pH greater than 5. In some embodiments, the orally disintegrating composition releases at least about 90% of the linaclotide contained therein within 30 seconds of contacting saliva having a pH greater than 5. In some embodiments, the orally disintegrating composition releases at least about 95% of the linaclotide contained therein within 30 seconds of contacting saliva having a pH greater than 5. In some embodiments, the orally disintegrating composition releases at least about 99% of the linaclotide contained therein within 30 seconds of contacting saliva having a pH greater than 5.

In some embodiments, the orally disintegrating composition releases at least about 75% of the linaclotide contained therein within 30 seconds of contacting phosphate buffer solution having a pH of 4.5 and maintained at 37±1° C. In some embodiments, the orally disintegrating composition releases at least about 80% of the linaclotide contained therein within 30 seconds of contacting phosphate buffer solution having a pH of 4.5 and maintained at 37±1° C. In some embodiments, the orally disintegrating composition releases at least about 85% of the linaclotide contained therein within 30 seconds of contacting phosphate buffer solution having a pH of 4.5 and maintained at 37±1° C. In some embodiments, the orally disintegrating composition releases at least about 90% of the linaclotide contained therein within 30 seconds of contacting phosphate buffer solution having a pH of 4.5 and maintained at 37±1° C. In some embodiments, the orally disintegrating composition releases at least about 95% of the linaclotide contained therein within 30 seconds of contacting phosphate buffer solution having a pH of 4.5 and maintained at 37±1° C. In some embodiments, the orally disintegrating composition releases at least about 99% of the linaclotide contained therein within 30 seconds of contacting phosphate buffer solution having a pH of 4.5 and maintained at 37±1° C.

The composition can also be used to treat and other diseases, disorders, or conditions that are responsive to treatment with agonists of the GC-C receptor. The composition can be used to treat any gastrointestinal disorders and/or conditions in a patient (e.g., mammal or human) or inflammation or pain associated therewith. Suitable such gastrointestinal disorders and conditions, include, but are not limited to, irritable bowel syndrome, constipation-predominant irritable bowel syndrome, dyspepsia (including functional dyspepsia or non-ulcer dyspepsia), gastrointestinal motility disorders, functional gastrointestinal disorders, gastroesophageal reflux disease (GERD), Crohn's disease, ulcerative colitis, inflammatory bowel disease, functional heartburn, gastroparesis, chronic intestinal pseudo-obstruction (or colonic pseudo-obstruction), and disorders and conditions associated with constipation, for example, chronic constipation, opioid induced constipation, post-surgical constipation (post-operative ileus), and constipation associated with neuropathic disorders or a combination of symptoms thereof (such as a combination of irritable bowel syndrome and chronic constipation). In some embodiments, a method is provided for treating gastrointestinal disorders in a patient (e.g., mammal or human) diagnosed with one or more gastrointestinal disorders or conditions, wherein the method comprises administering an effective amount of the composition to the patient.

In another embodiment, a method is provided for increasing intestinal motility in a patient in need thereof, comprising administering an effective amount of the composition to the patient. Intestinal motility involves spontaneous coordinated dissentions and contractions of the stomach, intestines, colon and rectum to move food through the gastrointestinal tract during the digestive process.

In exemplary embodiments, the methods may comprise administering a therapeutically effective amount of the pharmaceutical composition to a patient in need thereof.

An effective amount of a composition comprising linaclotide or a pharmaceutically acceptable salt thereof required to achieve desired results (such as desired treatment and/or symptom relief) of a subject is dependent on several understood factors, such as the identity and severity of the disorder being treated, as well as the age, weight, etc., of the patient being treated.

A subject or patient in whom administration of the pharmaceutical composition is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions described herein are particularly suited for administration to any animal, particularly a mammal, and including, but by no means limited to, humans, rodents and non-rodents, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use.

In some embodiments, the effective dose range of linaclotide for adult humans is from 25 μg to 6 mg per day orally. In some embodiments, the dose range is 25 μg to 2 mg per day orally. In some embodiments, the dose range for adult humans is 50 μg to 1 mg per day orally (e.g., 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg or 1 mg). In some embodiments, the dose range is 100 μg to 600 μg per day orally. In some embodiments, the dose is 50 μg, 100 μg, 150 μg, 200 μg, 300 μg, 400 μg, 500 μg or 600 μg linaclotide per day orally. In some embodiments, the dose is 50 μg linaclotide per day orally. In some embodiments, the dose is 100 μg linaclotide per day orally. In some embodiments, the dose is 150 μg linaclotide per day orally. In some embodiments, the dose is 200 μg linaclotide per day orally. In some embodiments, the dose is 300 μg linaclotide per day orally. In some embodiments, the dose is 400 μg linaclotide per day orally. In some embodiments, the dose is 500 μg linaclotide per day orally. In some embodiments, the dose is 600 μg linaclotide per day orally. In some embodiments, the effective pediatric dose range of linaclotide is from 0.05 μg to 2 mg per day orally. In some embodiments, the effective pediatric dose range of linaclotide is from 0.05 μg to 100 μg per day orally. In some embodiments, the effective pediatric dose range of linaclotide is from 0.1 μg to 90 μg per day orally. In some embodiments, the effective pediatric dose range of linaclotide is from 0.1 μg to 50 μg per day orally. In some embodiments, the effective pediatric dose range of linaclotide is from 0.1 μg to 25 μg per day orally. In some embodiments, the effective pediatric dose range of linaclotide is from 0.1 μg to 10 μg per day orally. In some embodiments, the effective pediatric dose range of linaclotide is from 0.1 μg to 5 μg per day orally. In some embodiments, the effective pediatric dose range of linaclotide is from 0.1 μg to 1 μg per day orally. In some embodiments, the effective pediatric dose range of linaclotide is from 0.1 μg to 0.5 μg per day orally. In some embodiments, the effective pediatric dose range of linaclotide is 0.1 μg per day orally. In some embodiments, the effective pediatric dose range of linaclotide is 0.15 μg per day orally. In some embodiments, the effective pediatric dose range of linaclotide is 0.25 μg per day orally. In some embodiments, the effective pediatric dose range of linaclotide is 0.5 μg per day orally. In some embodiments, the effective pediatric dose range of linaclotide is 3.5 μg per day orally. In some embodiments, the effective pediatric dose range of linaclotide is 15 μg per day orally. In some embodiments, the effective pediatric dose range of linaclotide is 45 μg per day orally. In some embodiments, the effective pediatric dose range of linaclotide is 60 μg per day orally. In some embodiments, the effective pediatric dose range of linaclotide is 90 μg per day orally. In some embodiments, the unit dosage form and daily dose are equivalent. In some embodiments, the unit dosage form is administered with food at anytime of the day, without food at anytime of the day, with food after an overnight fast (e.g., with breakfast). In some embodiments, the unit dosage form is administered once a day, twice a day or three times a day. In some embodiments, one, two or three unit dosage forms will contain the daily oral dose of linaclotide. The precise amount of compound administered to a patient will be the responsibility of the attendant physician. However, the dose employed will depend on a number of factors, including the age and sex of the patient, the precise disorder being treated, and its severity.

In some embodiments, the compositions are administered as a monotherapy. In some embodiments, the composition consists essentially of an effective amount of linaclotide. In some embodiments, the composition consists of an effective amount of linaclotide.

In some embodiments, the compositions are directly administered to a patient, for example, in the form of orally disintegrating tablet or orally disintegrating film. In some embodiments, the compositions are dissolved, disintegrated and/or mixed on or within food or beverage prior to administration to patients (e.g., elderly or pediatric patients). In some embodiments, the composition is dissolved or disintegrated in a liquid, solution, or fluid optionally containing stabilizing agent(s), preservative(s), sweetener(s), or the like, etc. prior to administration to a patient (e.g., elderly or pediatric patient). In some embodiments, the composition is a multiple dose composition, i.e., containing two, three, five, seven, ten, fifteen, twenty, twenty-five, thirty, forty, fifty, sixty, seventy, eighty, ninety or more daily doses of linaclotide. In some embodiments, one or more orally disintegrating tablets or films containing 3.5 μg of linaclotide are dissolved or disintegrated within a liquid, solution, or fluid to provide a composition that contains a five day supply of 0.5 μg of linaclotide dosages of the composition (“a five dose composition”) (see, for example, Example 18). In some embodiments, one or more orally disintegrating tablets or films containing 15 μg of linaclotide are dissolved or disintegrated within a liquid, solution, or fluid to provide a composition that contains a thirty day supply of 0.5 μg of linaclotide dosages of the composition (“a thirty dose composition”) (see, for example, Example 18). In some embodiments, one or more orally disintegrating tablets or films containing 45 μg of linaclotide are dissolved or disintegrated within a liquid, solution, or fluid to provide a composition that contains a ninety day supply of 0.5 μg of linaclotide dosages of the composition (“a ninety dose composition”) (see, for example, Example 18). In some embodiments, one or more orally disintegrating tablets or films containing 60 μg of linaclotide are dissolved or disintegrated within a liquid, solution, or fluid to provide a composition that contains a 120 day supply of 0.5 μg of linaclotide dosages of the composition (“a 120 dose composition”) (see, for example, Example 18). In some embodiments, one or more orally disintegrating tablets or films containing 90 μg of linaclotide are dissolved or disintegrated within a liquid, solution, or fluid to provide a composition that contains a 180 day supply of 0.5 μg of linaclotide dosages of the composition (“a 180 dose composition”) (see, for example, Example 18).

In other embodiments, the compositions are administered as part of a combination therapy. For example, a composition may be used in combination with other drugs or therapies that are used in the treatment, prevention, suppression, and/or amelioration of the diseases or conditions for which compounds of the invention are useful. The linaclotide can be co-administered or co-formulated with other medications. In one embodiment, the linaclotide composition can be co-administered with other medications used to treat gastrointestinal disorders including but not limited to acid suppressing agents such as Histamine-2 receptor agonists (H2As) and/or proton pump inhibitors (PPIs).

Such other drug(s) may be administered, by a route and in an amount commonly used therefore, contemporaneously or sequentially with a compound of the invention. When a compound of the present invention is used contemporaneously with one or more other drugs, a pharmaceutical unit dosage form containing such other drugs in addition to the compound of the invention may be employed. Accordingly, the pharmaceutical compositions of the present invention include those that also contain one or more other active components, in addition to a compound of invention.

Several methods can be used for evaluating the bioactivity of the linaclotide composition, including, but not limited to, immunoassays (e.g., enzyme-linked immunosorbent assay), radioimmuno assays, immunoradiometric assays, gel electrophoresis (e.g., SDS-PAGE), high performance liquid chromatography (HPLC), and/or high performance capillary electrophoresis (HPCE). In some embodiments, the bioactivity of the composition is assessed by a method comprising fixing linaclotide, incubating linaclotide with guanylate cyclase C (GCC), incubating GCC bound linaclotide with antibodies against GCC, incubating GCC antibody-bound linaclotide with fluorescently labeled antibodies against GCC antibodies, and detecting the linaclotide bound to the GCC antibodies by measuring the fluorescence intensity using a plate reader. The drug concentration can then be calculated based on the fluorescence reading of the solution.

For example, the bioactivity of the linaclotide compositions can be assessed and quantified using the following method, though other methods are available. The composition is added to a volumetric flask containing 60 ml of phosphate buffer having a pH of 4.5, and the flask is shaken for 60 minutes. 0.2 ml of the supernatant is then removed, and is added into one or more wells of a 96-well plate that is coated with GCC. The plate is sealed and incubated at 37° C. for 2 hr. At the end of incubation, the sample is removed and the plate is washed with phosphate buffered saline (PBS). The bound linaclotide is then incubated for 1 hour, at room temperature, with GCC (such as is available from Sigma-Aldrich Inc.) labeled with fluorescein isocyanate (FITC) in blocking buffer. After incubation, the well is washed with PBS. The fluorescence intensity of the end product is detected, for example, by using a plate reader. The linaclotide concentration is then calculated based on the fluorescence reading of the solution.

### DEFINITIONS

As used herein, unless otherwise indicated, the terms “ODF,” “orally disintegrating film” and “orally dissolving film” are used synonymously and mean that the film dissolves, melts, disintegrates, liquefies, etc. in the oral cavity such that substantially all of the linaclotide no longer remains in a formulation form.

As used herein, unless otherwise indicated, the terms “ODT,” “orally disintegrating tablet” and “orally dissolving tablet” are used synonymously and mean that the film dissolves, melts; disintegrates, liquefies, etc. in the oral cavity such that substantially all of the linaclotide no longer remains in a formulation form.

As used herein, unless otherwise indicated, the “disintegration rate” is used herein to mean the amount of time that the film or tablet dissolves, melts, disintegrates, liquefies, etc. in the environment of an oral cavity such that substantially all of the linaclotide no longer remains in a formulation form, e.g., in saliva having a pH greater than 5, or in a phosphate buffer solution having a pH of 4.5 and maintained at 37±1° C.

As used herein, unless otherwise indicated, the term “entry into a use environment” means contact of the composition with saliva of the patient to whom it is administered, or with a fluid intended to simulate saliva, e.g., having a pH greater than 5, or with a phosphate buffer solution having a pH of 4.5 and maintained at 37±1° C.

The term “released from”, when referring to the release of linaclotide from the composition, unless otherwise indicated, is used herein to mean that the linaclotide no longer remains in a composition form.

As used herein, unless otherwise indicated, “stabilizing agent” refers to a polymer, sterically hindered primary amine (e.g., amino acid), or cation (e.g., metal cation) component of the composition which is included in the composition in a stabilizing amount. For example, a polymeric stabilizing agent is a polymer that is included in the composition in a stabilizing amount. Similarly, a sterically hindered primary amine stabilizing agent is a sterically hindered primary amine that is included in the composition in a stabilizing amount. Moreover, a cationic stabilizing agent is a cation that is included in the composition in a stabilizing amount.

As used herein, unless otherwise indicated, “stabilizing amount” refers to a concentration, within the composition, of a polymer, sterically hindered primary amine (e.g., amino acid), or metal cation component at which the component increases the stability of linaclotide in the composition, as compared to a similar composition not having a stabilizing amount of the same component.

As used herein, unless otherwise indicated, the term “substantially all” means at least about 90%, for example, at least about 95% or even at least about 99%.

As used herein, unless otherwise indicated, the term “isolated and purified” means at least 95 percent pure (for example, at least 96% pure, at least 97% pure, at least 98% pure, or even at least 99% pure), as measured, for example, by chromatographic purity using HPLC.

As used herein, unless otherwise indicated, “therapeutically effective amount” means the amount of a linaclotide or a pharmaceutically acceptable salt thereof that, when administered to a mammal for treating a state, disorder or condition, is sufficient to effect a treatment (as defined below). The “therapeutically effective amount” will vary depending on the compound, the disease and its severity and the age, sex, weight, physical condition and responsiveness of the mammal to be treated. For example, a therapeutically effective amount of linaclotide, or its pharmaceutically acceptable salt or hydrate, can be an amount effective to treat gastrointestinal disorders, including irritable bowel syndrome, constipation-predominant irritable bowel syndrome, chronic constipation, opioid induced constipation and/or dyspepsia.

As used herein, unless other indicated, “pharmaceutically acceptable” means biologically or pharmacologically compatible for in vivo use in animals or humans, and preferably means, approved by a regulatory agency of the Federal or a state government or listed in the U.S. Pharmacopeia or other generally recognized pharmacopeia for use in animals, and more particularly in humans.

As used herein, unless otherwise indicated, the term “treat”, in all its verb forms, is used herein to mean to relieve, alleviate, prevent, and/or manage at least one symptom of a disorder in a subject, the disorder including, for example, a gastrointestinal disorder, such as, irritable bowel syndrome, constipation-predominant irritable bowel syndrome, chronic constipation, opioid induced constipation, dyspepsia, or a combination of symptoms thereof. Within the meaning of the present invention, the term “treat” also denotes, to arrest, delay the onset (i.e., the period prior to clinical manifestation of a disease) and/or reduce the risk of developing or worsening a disease. The term “treatment” means the act of “treating” as defined above.

As used herein, unless otherwise indicated, the term “additives” refers to a pharmaceutically acceptable additive. Pharmaceutically acceptable additives include, without limitation, binders, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes.

As used herein, unless otherwise indicated, an “excipient” is any pharmaceutically acceptable additive, filler, binder or agent.

As used herein, unless otherwise indication, “stressed conditions” refer to 40° C. and 75% relative humidity (RH).

As used here, unless otherwise indicated, the terms “about” and “approximately” mean within an acceptable error range for the particular value as determined by one of ordinary skill in the art, which will depend, in part, on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%. Alternatively, particularly with respect to biological systems or processes, the term can mean within an order of magnitude, preferably within 5-fold, and more preferably within 2-fold, of a value. Particular values are described in the application and claims, unless otherwise stated the term “about” means within an acceptable error range for the particular value.

All weight percentages (i.e., “% by weight” and “wt. %” and w/w) referenced herein, unless otherwise indicated, are measured relative to the total weight of the pharmaceutical composition.

The term “consisting essentially of”, and variants thereof, when used to refer to the composition, are used herein to mean that the composition includes linaclotide and other desired pharmaceutically inactive additives, excipients, and/or components (e.g., polymers, sterically hindered primary amines, cations, filling agents, binders, carriers, excipients, diluents, disintegrating additives, lubricants, solvents, dispersants, coating additives, absorption promoting additives, hydrolysis products, formaldehyde imine products, oxidation products, acetylation products, deamidation products, multimers, controlled release additives, anti-caking additives, anti-microbial additives, preservatives, sweetening additives, colorants, flavors, desiccants, plasticizers, dyes, or the like), and no other active pharmaceutical ingredient(s).

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

The following tests were employed in the examples section, unless otherwise indicated:

1) Stability of Linaclotide Compositions.

For stability evaluation, linaclotide compositions (0.15 mg theoretical, actual 0.135 mg) were packaged into a HDPE bottle with desiccant, and stored under at 40° C./75% RH (“stressed conditions”). The amount of linaclotide was assayed initially and after 3, 6, 9, 12, or 18 months of storage at stressed conditions. The concentration of linaclotide was analyzed and quantified using an HPLC method with the following mobile phase gradient: Mobile phase A: 50 mM of sodium perchlorate in a solvent containing 76% water and 24% acetonitrile and 0.1% of trifluoroacetic acid; Mobile phase B: 50 mM of sodium perchlorate in a solvent containing 5% water and 95% acetonitrile and 0.1% of trifluoroacetic acid; Flow rate: 0.6 ml/min; Column: YMC Pro C18, 150 mm×3 mm ID, 3 μm or equivalent; Column temperature: 40° C.; Fluorescence detection: excitation: 274 nm; emission: 303 nm; Injection volume: 100 μl.

2) Analysis of Total Degradants in the Pharmaceutical Composition:

Degradant analysis was performed using an HPLC method employing the following conditions: Mobile phase A: Water:acetonitrile 98: 2, with 0.1% (v/v) of trifluoroacetic acid; Mobile phase B: Water: acetonitrile 5: 95, with 0.1% (v/v) of trifluoroacetic acid; Flow rate: 0.6 ml/min; Column: YMC Pro C18, 150 mm×3 mm ID, 3 μm or equivalent; Column temperature: 40° C.; UV detection: excitation: 220 nm; Injection volume: 50 μl. The percentage amounts of degradants in the composition were calculated by quantifying the area of all peaks in the HPLC chromatogram to obtain the “total peak area”, and dividing the peak area of each degradant by the total peak area.

3) Dissolution Test:

The dissolution performance of the composition was assessed in phosphate buffer, pH 4.5 using USP Apparatus II (Paddle, 50 rpm).

4) Disintegration Test:

The disintegration of orally disintegrating compositions of linaclotide was performed in a USP standard disintegrating test apparatus. The disintegration medium utilized was phosphate buffer, pH 4.5 maintained at 37±1 C.°. Mean disintegration time was calculated by averaging the disintegration time of six orally disintegrating compositions (e.g., tablets) of linaclotide.

### Example 1

### Orally Disintegrating IR Tablet Comprising Linaclotide

An orally disintegrating tablet comprising linaclotide was prepared in the following manner. PVP was dissolved in citric buffer (20 mM, pH 3) with citric acid and sodium citrate, while stirring, until a clear solution was obtained. Calcium chloride, leucine and mannitol were then dissolved in the PVP-citric buffer solution, while stirring, until a clear solution was obtained. Half of the PVP-citric buffer solution was removed to a container and linaclotide was dissolved in the solution, while stirring, until a clear linaclotide solution was obtained. The other half of the PVP-citric buffer solution was heated in a water bath (60° C.), and gelatin was dissolved in the solution until a clear solution was obtained. The gelatin solution was cooled to room temperature. The clear linaclotide solution was then added to the gelatin solution and the combination was mixed until a clear solution was obtained. The composition was then placed into the cavities of an aluminum blister, with approximately 0.6 ml of solution in each cavity. The solution-containing blisters were then frozen at −20° C. overnight, followed by deep freezing in a dry ice-acetone solution. The blisters were then lyophilized in a lyophilizer (−52° C., 0.5 Torr) overnight. The lyophilized tablets were placed into aluminum pouches, and were the pouches were sealed.

Tables 1 and 2 illustrate oral disintegrating tablets of linaclotide that were produced in this manner.

TABLE 1
Linaclotide oral disintegrating tablet, 0.15 mg/90 mg

| | Weight/tablet | Theoretical Weight |
|---|---|---|
| Components | (mg) | mg/g |
| Linaclotide | 0.15 | 1.7 |
| Mannitol | 70.6 | 784 |
| Calcium chloride dihydrate | 0.95 | 10.6 |
| Leucine | 0.42 | 4.7 |
| PVP | 18 | 200 |
| Purified water, USP* | — | — |
| Total | 90.1 | 1000 |

*Water is removed during the manufacturing process

TABLE 2
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 180 | 300 | 600 | 900 |
| Linaclotide | 0.001 | 0.01 | 0.025 | 0.05 | 0.075 | 0.15 | 0.18 | 0.3 | 0.6 | 0.9 |
| Mannitol | 90 | 88.7 | 86.7 | 83.5 | 80.2 | 70.6 | 66.6 | 111 | 71.9 | 33 |
| Calcium chloride | 0.006 | 0.064 | 0.16 | 0.32 | 0.475 | 0.95 | 1.14 | 1.9 | 3.8 | 5.7 |
| dihydrate | | | | | | | | | | |
| Leucine | 0.003 | 0.028 | 0.07 | 0.14 | 0.21 | 0.42 | 0.5 | 0.84 | 1.68 | 2.52 |
| PVP | 0.12 | 1.2 | 3 | 6 | 9 | 18 | 21.6 | 36 | 72 | 108 |
| Purified water, | — | — | — | — | — | — | — | — | — | — |
| USP* | | | | | | | | | | |
| Total (mg) | 90 | 90 | 90 | 90 | 90 | 90.1 | 90 | 150 | 150 | 150 |

*Water is removed during the manufacturing process

The stability, dissolution, and disintegration performance of the oral disintegrating tablet defined in Table 1 was assessed, as is illustrated in Table 3.

TABLE 3
Stability, Dissolution, and Disintegration Performance of Oral
Disintegrating Tablet (0.15 mg/90 mg) in aluminum pouch,
with 2 g desiccant

| | | Total | | | | |
|---|---|---|---|---|---|---|
| | Desiccant | Linaclotide | Dissolution % | | | Disintegration |
| Condition | (g) | (mcg) | 1 min | 5 min | Total Deg % | time |
| Initial | N/A | 131 | 95.6 | 97.5 | 1.29 | 2 sec |
| 40/75, | 2 | 123 | 94.1 | 94.6 | 2.48 | 2 sec |
| 1 month | | | | | | |
| 40/75, | 2 | 131 | 95.7 | 100 | 3.82 | 2 sec |
| 2 months | | | | | | |

### Example 2

### Orally Disintegrating IR Tablet Comprising Linaclotide

Orally disintegrating linaclotide tablets comprising components as shown in Tables 4 and 5 were prepared in the manner described in Example 1. The stability, dissolution, and disintegration performance of the oral disintegrating tablets (0.15 mg/90 mg, in aluminum pouch, with 2 g desiccant) was assessed, as is illustrated in Table 6.

TABLE 4
Linaclotide oral disintegrating tablet, 0.15 mg/90 mg

| | Weight/tablet | Theoretical Weight |
|---|---|---|
| Components | (mg) | Mg/g |
| Linaclotide | 0.15 | 1.7 |
| Mannitol | 30.9 | 343 |
| Calcium chloride dihydrate | 0.6 | 6.7 |
| PVP | 18.3 | 206 |
| Gelatin | 37.3 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 5
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 180 | 300 | 600 | 900 |
| Linaclotide | 0.001 | 0.01 | 0.025 | 0.05 | 0.075 | 0.15 | 0.18 | 0.3 | 0.6 | 0.9 |
| Mannitol | 89.6 | 86.1 | 80.2 | 70.5 | 60 | 30.9 | 18.59 | 31.9 | 30.4 | 28.9 |
| Calcium chloride | 0.004 | 0.04 | 0.1 | 0.2 | 0.3 | 0.6 | 0.73 | 1.2 | 2.4 | 3.6 |
| dihydrate | | | | | | | | | | |
| PVP | 0.122 | 1.22 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium citrate | 0.004 | 0.04 | 0.1 | 0.17 | 0.25 | 0.5 | 0.5 | 1 | 1 | 1 |
| Purified water, | — | — | — | — | — | — | — | — | — | — |
| USP* | | | | | | | | | | |
| Total (mg) | 90 | 90 | 90 | 90 | 90 | 90 | 90 | 150 | 150 | 150 |

*Water is removed during the manufacturing process

TABLE 6
Performance of Oral Disintegrating Tablet

| | | Total | | | |
|---|---|---|---|---|---|
| | Desiccant | Linaclotide | Dissolution % | | Disintegration |
| Condition | G | (mcg) | 1 min | 5 min | time |
| Initial | N/A | 183 | 33.6 | 104 | 3 min |

### Example 3

Orally disintegrating linaclotide tablets comprising components as shown in Tables 7 and 8 were prepared in the manner described in Example 1. The stability, dissolution, and disintegration performance of the orally disintegrating linaclotide tablets (0.15 mg/90 mg, in aluminum pouch, with 2 g desiccant) were evaluated as is illustrated in Table 9.

TABLE 7
Linaclotide oral disintegrating tablet, 0.15 mg/90 mg

| | Weight/tablet | Theoretical Weight |
|---|---|---|
| Components | (mg) | mg/g |
| Linaclotide | 0.15 | 1.7 |
| Mannitol | 30.9 | 343 |
| Calcium chloride dehydrate | 0.6 | 6.7 |
| PVP | 18.3 | 206 |
| Gelatin | 37.3 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 8
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 215 | 300 | 600 | 900 |
| linaclotide | 0.001 | 0.01 | 0.025 | 0.05 | 0.75 | 0.15 | 0.215 | 0.3 | 0.6 | 0.9 |
| Mannitol | 90 | 88.5 | 86.2 | 82.35 | 77.6 | 68 | 60 | 112.9 | 111.4 | 109.9 |
| Calcium | 0.004 | 0.04 | 0.1 | 0.2 | 0.3 | 0.6 | 0.8 | 1.2 | 2.4 | 3.6 |
| chloride | | | | | | | | | | |
| dihydrate | | | | | | | | | | |
| PVP | 0.13 | 1.3 | 3.25 | 6.5 | 10 | 18.5 | 26.5 | 31 | 31 | 31 |
| Citric acid, | 0.014 | 0.14 | 0.35 | 0.7 | 1.1 | 2.2 | 2.2 | 3.7 | 3.7 | 3.7 |
| anhydrous | | | | | | | | | | |
| Sodium | 0.004 | 0.04 | 0.1 | 0.2 | 0.25 | 0.5 | 0.5 | 0.9 | 0.9 | 0.9 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 90 | 150 | 150 | 150 |

*Water is removed during the manufacturing process

TABLE 9
Stability, Dissolution, and Disintegration Performance
of Oral Disintegrating Tablet (0.15 mg/90 mg)
in aluminum pouch with 2 g desiccant

| | Total | | | | |
|---|---|---|---|---|---|
| | Linaclotide | Total | Dissolution % | | |
| Condition | (mcg) | Deg % | 1 min | 5 min | Disintegration time |
| Initial | 140 | 1.29 | 95.6 | 97.5 | 2 sec |
| 40/75, | 138.2 | 2.48 | 100 | 100 | 2 sec |
| 1 month | | | | | |

### Example 4

Orally disintegrating linaclotide tablets comprising components as shown in Tables 10 and 11 may be prepared as described in Example 1 using PVA as stabilizing agent.

TABLE 10
Linaclotide oral disintegrating tablet, 150 mcg/90 mg

| | Weight/tablet | Theoretical Weight |
|---|---|---|
| Components | (mg) | mg/g |
| Linaclotide | 0.15 | 1.7 |
| Mannitol | 68 | 755 |
| Calcium chloride dihydrate | 0.6 | 6.7 |
| PVA | 18.5 | 206 |
| Citric acid, anhydrous | 2.2 | 24.6 |
| Sodium citrate | 0.5 | 5.8 |
| Purified water, USP* | — | — |
| Total | | 1000 |

*Water is removed during the manufacturing process

TABLE 11
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 90 | 88.5 | 86.2 | 82.35 | 77.6 | 68 | 112.9 | 111.4 | 109.9 | 109.6 |
| Calcium | 0.004 | 0.04 | 0.1 | 0.2 | 0.3 | 0.6 | 1.2 | 2.4 | 3.6 | 3.6 |
| chloride | | | | | | | | | | |
| dihydrate | | | | | | | | | | |
| .PVA | 0.13 | 1.3 | 3.25 | 6.5 | 10 | 18.5 | 31 | 31 | 31 | 31 |
| Citric acid, | 0.014 | 0.14 | 0.35 | 0.7 | 1.1 | 2.2 | 3.7 | 3.7 | 3.7 | 3.7 |
| anhydrous | | | | | | | | | | |
| Sodium | 0.004 | 0.04 | 0.1 | 0.2 | 0.25 | 0.5 | 0.9 | 0.9 | 0.9 | 0.9 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 150 | 150 | 150 | 150 |

*Water is removed during the manufacturing process

### Example 5

Orally disintegrating linaclotide IR tablets comprising components as shown in Tables 12 and 13 may be prepared as described in Example 1 using sucrose as stabilizing agent.

TABLE 12
Linaclotide oral disintegrating tablet, 150 mcg/90 mg

| | Weight/tablet | Theoretical Weight |
|---|---|---|
| Components | (mg) | mg/g |
| Linaclotide | 0.15 | 1.7 |
| Mannitol | 68 | 755 |
| Calcium chloride dihydrate | 0.6 | 6.7 |
| Sucrose | 18.5 | 206 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 13
Linaclotide oral disintegrating tablet of various strengths

| | Tablet composition (mg/g) of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 90 | 88.5 | 86.2 | 82.35 | 77.6 | 68 | 112.9 | 111.4 | 109.9 | 109.6 |
| Calcium | 0.004 | 0.04 | 0.1 | 0.2 | 0.3 | 0.6 | 1.2 | 2.4 | 3.6 | 3.6 |
| chloride | | | | | | | | | | |
| dihydrate | | | | | | | | | | |
| Sucrose | 0.13 | 1.3 | 3.25 | 6.5 | 10 | 18.5 | 31 | 31 | 31 | 31 |
| Citric acid, | 0.014 | 0.14 | 0.35 | 0.7 | 1.1 | 2.2 | 3.7 | 3.7 | 3.7 | 3.7 |
| anhydrous | | | | | | | | | | |
| Sodium | 0.004 | 0.04 | 0.1 | 0.2 | 0.25 | 0.5 | 0.9 | 0.9 | 0.9 | 0.9 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 150 | 150 | 150 | 150 |

*Water is removed during the manufacturing process

### Example 6

Orally disintegrating linaclotide IR tablets comprising components as shown in Tables 14 and 15 may be prepared as described in Example 1 using sucrose as stabilizing agent.

TABLE 14
Linaclotide oral disintegrating tablet, 150 mcg/90 mg

| | Weight/tablet | Theoretical Weight |
|---|---|---|
| Components | (mg) | mg/g |
| Linaclotide | 0.15 | 1.7 |
| Mannitol | 31 | 343 |
| Calcium chloride dihydrate | 0.6 | 6.7 |
| sucrose | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 15
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| Calcium | 0.004 | 0.04 | 0.1 | 0.2 | 0.3 | 0.6 | 0.73 | 1.2 | 2.4 | 3.6 |
| chloride | | | | | | | | | | |
| dihydrate | | | | | | | | | | |
| sucrose | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total (mcg) | 90 | 90 | 90 | 90 | 90 | 90 | 90 | 150 | 150 | 150 |

*Water is removed during the manufacturing process

### Example 7

Orally disintegrating linaclotide IR tablets comprising components as shown in Tables 16 and 17 may be prepared as described in Example 1 using cyclodextrin as stabilizing agent.

TABLE 16
Linaclotide oral disintegrating tablet, 150 mcg/90 mg

| | Weight/tablet | Theoretical Weight |
|---|---|---|
| Components | (mg) | mg/g |
| Linaclotide | 0.15 | 1.7 |
| Mannitol | 31 | 343 |
| Calcium chloride dihydrate | 0.6 | 6.7 |
| HP-β-CD | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 17
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| Calcium | 0.004 | 0.04 | 0.1 | 0.2 | 0.3 | 0.6 | 0.73 | 1.2 | 2.4 | 3.6 |
| chloride | | | | | | | | | | |
| dihydrate | | | | | | | | | | |
| HP-β-CD | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total | 90 | 90 | 90 | 90 | 90 | 90 | 90 | 150 | 150 | 150 |

*Water is removed during the manufacturing process

### Example 8

Orally disintegrating linaclotide IR tablets comprising components as shown in Tables 18 and 19 may be prepared as described in Example 1 using dextrin as stabilizing agent.

TABLE 18
Linaclotide oral disintegrating tablet, 150 mcg/90 mg

| | Weight/tablet | Theoretical Weight |
|---|---|---|
| Components | (mg) | mg/g |
| Linaclotide | 0.15 | 1.7 |
| Mannitol | 31 | 343 |
| Calcium chloride dihydrate | 0.6 | 6.7 |
| dextrin | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 19
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| Calcium | 0.004 | 0.04 | 0.1 | 0.2 | 0.3 | 0.6 | 0.73 | 1.2 | 2.4 | 3.6 |
| chloride | | | | | | | | | | |
| dihydrate | | | | | | | | | | |
| dextrin | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 90 | 150 | 150 | 150 |

*Water is removed during the manufacturing process

### Example 9

Orally disintegrating linaclotide IR tablets comprising components as shown in Tables 20 and 21 may be prepared as described in Example 1 using xanthan as stabilizing agent.

TABLE 20
Linaclotide oral disintegrating tablet, 0.15 mg/90 mg

| | Weight/tablet | Theoretical Weight |
|---|---|---|
| Components | (mg) | mg/g |
| Linaclotide | 0.15 | 1.7 |
| Mannitol | 31 | 343 |
| Calcium chloride dihydrate | 0.6 | 6.7 |
| xanthan | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 21
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| Calcium | 0.004 | 0.04 | 0.1 | 0.2 | 0.3 | 0.6 | 0.73 | 1.2 | 2.4 | 3.6 |
| chloride | | | | | | | | | | |
| dihydrate | | | | | | | | | | |
| xanthan | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 90 | 150 | 150 | 150 |

*Water is removed during the manufacturing process

### Example 10

Orally disintegrating linaclotide IR tablets comprising components as shown in Tables 22 and 23 may be prepared as described in Example 1 using trehalose as stabilizing agent.

TABLE 22
Linaclotide oral disintegrating tablet, 0.15 mg/90 mg

| | | Theoretical |
|---|---|---|
| | Weight/tablet | Weight |
| Components | (mg) | mg/g |
| Linaclotide | 0.15 | 1.7 |
| Mannitol | 31 | 343 |
| Calcium chloride dihydrate | 0.6 | 6.7 |
| trehalose | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 23
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| Calcium | 0.004 | 0.04 | 0.1 | 0.2 | 0.3 | 0.6 | 0.73 | 1.2 | 2.4 | 3.6 |
| chloride | | | | | | | | | | |
| dihydrate | | | | | | | | | | |
| trehalose | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 90 | 150 | 150 | 150 |

*Water is removed during the manufacturing process

### Example 11

Orally disintegrating linaclotide IR tablets comprising components as shown in Tables 24 and 25 may be prepared as described in Example 1 using sodium chloride as stabilizing agent.

TABLE 24
Linaclotide oral disintegrating tablet, 0.15 mg/90 mg

| | | Theoretical |
|---|---|---|
| | Weight/tablet | Weight |
| Components | (mg) | mg/g |
| Linaclotide | 0.15 | 1.7 |
| Mannitol | 31 | 343 |
| Sodium chloride | 0.6 | 6.7 |
| PVP | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 25
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| Sodium | 0.004 | 0.04 | 0.1 | 0.2 | 0.3 | 0.6 | 0.73 | 1.2 | 2.4 | 3.6 |
| chloride | | | | | | | | | | |
| PVP | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 90 | 150 | 150 | 150 |

*Water is removed during the manufacturing process

### Example 12

Orally disintegrating linaclotide IR tablets comprising components as shown in Tables 26 and 27 may be prepared as described in Example 1 using glycine as stabilizing agent.

TABLE 26
Linaclotide oral disintegrating tablet, 0.15 mg/90 mg

| | | Theoretical |
|---|---|---|
| | Weight/tablet | Weight |
| Components | (mg) | mg/g |
| Linaclotide | 0.15 | 1.7 |
| Mannitol | 31 | 343 |
| glycine | 0.6 | 6.7 |
| PVP | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 27
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| glycine | 0.004 | 0.04 | 0.1 | 0.2 | 0.3 | 0.6 | 0.73 | 1.2 | 2.4 | 3.6 |
| PVP | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 90 | 150 | 150 | 150 |

*Water is removed during the manufacturing process

### Example 13

Orally disintegrating linaclotide IR tablets comprising components as shown in Tables 28 and 29 may be prepared as described in Example 1 using leucine as stabilizing agent.

TABLE 28
Linaclotide oral disintegrating tablet, 0.15 mg/90 mg

| | Weight/tablet | Theoretical Weight |
|---|---|---|
| Components | (mg) | mg/g |
| Linaclotide | 0.15 | 1.7 |
| Mannitol | 31 | 343 |
| leucine | 0.6 | 6.7 |
| PVP | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 29
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| leucine | 0.004 | 0.04 | 0.1 | 0.2 | 0.3 | 0.6 | 0.73 | 1.2 | 2.4 | 3.6 |
| PVP | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 90 | 150 | 150 | 150 |

*Water is removed during the manufacturing process

### Example 14

Orally disintegrating linaclotide IR tablets comprising components as shown in Table 30 and 31 may be prepared as described in Example 1 using inulin as stabilizing agent.

TABLE 30
Linaclotide oral disintegrating tablet, 0.15 mg/90 mg

| | | Theoretical |
|---|---|---|
| | Weight/tablet | Weight |
| Components | (mg) | mg/g |
| Linaclotide | 0.15 | 1.7 |
| Mannitol | 31 | 343 |
| Calcium chloride | 0.6 | 6.7 |
| Inulin | 18.5 | 206 |
| Gelatin | 37 | 414 |
| Citric acid, anhydrous | 2.2 | 24.6 |
| Sodium citrate | 0.5 | 5.8 |
| Purified water, USP* | — | — |
| Total | 90 | 1000 |

*Water is removed during the manufacturing process

TABLE 31
Linaclotide oral disintegrating tablet of various strengths

| | Tablet composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| Linaclotide | 0.001 | 0.01 | 0.025 | 0.05 | 0.75 | 0.15 | 0.3 | 0.6 | 0.9 | 1.2 |
| Mannitol | 89.6 | 86 | 81 | 70.5 | 60 | 30.9 | 18.59 | 31.9 | 30.4 | 28.9 |
| Calcium | 0.004 | 0.04 | 0.1 | 0.2 | 0.3 | 0.6 | 0.73 | 1.2 | 2.4 | 3.6 |
| chloride | | | | | | | | | | |
| Inulin | 0.13 | 1.3 | 3.05 | 6.1 | 9.2 | 18.3 | 22.3 | 36.6 | 36.6 | 36.6 |
| Gelatin | 0.248 | 2.48 | 6.2 | 12.4 | 19 | 37.3 | 45.5 | 74.6 | 74.6 | 74.6 |
| Citric acid, | 0.014 | 0.14 | 0.35 | 0.7 | 1.1 | 2.2 | 2.2 | 4.4 | 4.4 | 4.4 |
| anhydrous | | | | | | | | | | |
| Sodium | 0.004 | 0.04 | 0.1 | 0.17 | 0.25 | 0.5 | 0.5 | 1 | 1 | 1 |
| citrate | | | | | | | | | | |
| Purified | — | — | — | — | — | — | — | — | — | — |
| water, USP* | | | | | | | | | | |
| Total (mg) | 90 | 90 | 90 | 90 | 90 | 90 | 90 | 150 | 150 | 150 |

*Water is removed during the manufacturing process

### Example 15

### Linaclotide Oral Disintegrating Film (ODF)

An orally disintegrating film comprising linaclotide was prepared by dissolving polyvinyl pyrrolidone (PVP) in solvent (water, ethanol, isopropanol, or their mixture) followed by the addition of plasticizer (polyethylene glycol), sweetener (Thaumatin, Acesulfan K), flavoring agent (orange, lemon, or cherry powder). Linaclotide, on the other hand, is dissolved in water together with leucine and calcium chloride dihydrate. The linaclotide solution was then added to the polymer solution and mixed for 30 minutes. The film was prepared by casting the drug/polymer solution onto a Teflon-coated surface and spread using a BYK-Gardner film casting knife followed by drying in oven at 50° C. for 1 h. The dried film is weighed and cut into the size so that each piece contains a dose ranging from 75 to 1200 mcg.

Table 32 illustrates the composition of an orally disintegrating film of linaclotide

TABLE 32
Linaclotide oral disintegrating film

| | Amount per film | |
|---|---|---|
| Ingredient | (mg) | w/w % |
| Linaclotide | 0.15 | 0.16 |
| PVP k90 | 60 | 67.8 |
| Polyethylene glycol | 12 | 13.6 |
| 400 | | |
| Leucine | 0.4 | 0.45 |
| Calcium chloride | 0.9 | 1.0 |
| Thaumatin | 5 | 5.6 |
| Water/Ethanol* | Q.S. | Q.S. |
| Orange powder | 10 | 11.3 |
| Total weight | 88.5 | 100 |

*Solvent is removed during the manufacturing process

TABLE 33
Linaclotide oral disintegrating film of various strengths

| | Film composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| Linaclotide | 0.001 | 0.01 | 0.025 | 0.05 | 0.75 | 0.15 | 0.3 | 0.6 | 0.9 | 1.2 |
| PVP k90 | 60 | 60 | 60 | 60 | 30 | 60 | 120 | 240 | 360 | 480 |
| Polyethylene | 12 | 12 | 12 | 12 | 6 | 12 | 24 | 48 | 72 | 96 |
| glycol 400 | | | | | | | | | | |
| Leucine | 0.003 | 0.03 | 0.075 | 0.15 | 0.225 | 0.4 | 0.8 | 1.6 | 2.4 | 3.2 |
| Calcium | 0.006 | 0.06 | 0.15 | 0.3 | 0.45 | 0.9 | 1.8 | 3.6 | 5.4 | 7.2 |
| chloride | | | | | | | | | | |
| Thaumatin | 5 | 5 | 5 | 5 | 2.5 | 5 | 10 | 20 | 30 | 40 |
| Water/Ethanol | Q.S. | Q.S. | Q.S. | Q.S. | Q.S. | Q.S. | Q.S. | Q.S. | Q.S. | Q.S. |
| * | | | | | | | | | | |
| Orange | 10 | 10 | 10 | 10 | 5 | 10 | 20 | 30 | 40 | 50 |
| powder | | | | | | | | | | |
| Total (mg) | 87 | 87.1 | 87.25 | 87.5 | 45 | 88.5 | 177 | 354 | 531 | 708 |

* Water is removed during the manufacturing process

### Example 16

An orally disintegrating linaclotide film comprising components as shown in Tables 34-35 may be prepared as described in Example 15.

TABLE 34
Linaclotide oral disintegrating film

| | Amount per film | |
|---|---|---|
| Ingredient | (mg) | w/w % |
| Linaclotide | 0.15 | 0.16 |
| polyvinyl alcohol | 60 | 67.8 |
| (PVA) | | |
| Glycerol | 12 | 13.6 |
| Leucine | 0.4 | 0.45 |
| Calcium chloride | 0.9 | 1.0 |
| Thaumatin | 5 | 5.6 |
| Water/Ethanol * | Q.S. | Q.S. |
| Orange powder | 10 | 11.3 |
| Total weight | 88.5 | 100 |

TABLE 35
Linaclotide oral disintegrating film of various strengths

| | Film composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| Linaclotide | 0.001 | 0.01 | 0.025 | 0.05 | 0.75 | 0.15 | 0.3 | 0.6 | 0.9 | 1.2 |
| PVA | 60 | 60 | 60 | 60 | 30 | 60 | 120 | 240 | 360 | 480 |
| Glycerol | 12 | 12 | 12 | 12 | 6 | 12 | 24 | 48 | 72 | 96 |
| Leucine | 0.003 | 0.03 | 0.075 | 0.15 | 0.225 | 0.4 | 0.8 | 1.6 | 2.4 | 3.2 |
| Calcium | 0.006 | 0.06 | 0.15 | 0.3 | 0.45 | 0.9 | 1.8 | 3.6 | 5.4 | 7.2 |
| chloride | | | | | | | | | | |
| Thaumatin | 5 | 5 | 5 | 5 | 2.5 | 5 | 10 | 20 | 30 | 40 |
| Water/Ethanol | Q.S. | Q.S. | Q.S. | Q.S. | Q.S. | Q.S. | Q.S. | Q.S. | Q.S. | Q.S. |
| * | | | | | | | | | | |
| Orange | 10 | 10 | 10 | 10 | 5 | 10 | 20 | 30 | 40 | 50 |
| powder | | | | | | | | | | |
| Total (mg) | 87 | 87.1 | 87.25 | 87.5 | 45 | 88.5 | 177 | 354 | 531 | 708 |

### Example 17

An orally disintegrating linaclotide film comprising components as shown in Tables 36-37 may be prepared as described in Example 15.

TABLE 36
Linaclotide oral disintegrating film

| | Amount per film | |
|---|---|---|
| Ingredient | (mg) | w/w % |
| Linaclotide | 0.15 | 0.16 |
| Carbopol | 60 | 67.8 |
| Glycerol | 12 | 13.6 |
| Leucine | 0.4 | 0.45 |
| Calcium chloride | 0.9 | 1.0 |
| Thaumatin | 5 | 5.6 |
| Water/Ethanol * | Q.S. | Q.S. |
| Orange powder | 10 | 11.3 |
| Total weight | 88.5 | 100 |

TABLE 37
Linaclotide oral disintegrating film of various strengths

| | Film composition of strength (mcg) | | | | | | | | | |
|---|---|---|---|---|---|---|---|---|---|---|
| Components | 1 | 10 | 25 | 50 | 75 | 150 | 300 | 600 | 900 | 1200 |
| Linaclotide | 0.001 | 0.01 | 0.025 | 0.05 | 0.75 | 0.15 | 0.3 | 0.6 | 0.9 | 1.2 |
| Carpol | 60 | 60 | 60 | 60 | 30 | 60 | 120 | 240 | 360 | 480 |
| Glycerol | 12 | 12 | 12 | 12 | 6 | 12 | 24 | 48 | 72 | 96 |
| Leucine | 0.003 | 0.03 | 0.075 | 0.15 | 0.225 | 0.4 | 0.8 | 1.6 | 2.4 | 3.2 |
| Calcium | 0.006 | 0.06 | 0.15 | 0.3 | 0.45 | 0.9 | 1.8 | 3.6 | 5.4 | 7.2 |
| chloride | | | | | | | | | | |
| Thaumatin | 5 | 5 | 5 | 5 | 2.5 | 5 | 10 | 20 | 30 | 40 |
| Water/Ethanol | Q.S. | Q.S. | Q.S. | Q.S. | Q.S. | Q.S. | Q.S. | Q.S. | Q.S. | Q.S. |
| * | | | | | | | | | | |
| Orange | 10 | 10 | 10 | 10 | 5 | 10 | 20 | 30 | 40 | 50 |
| powder | | | | | | | | | | |
| Total (mg) | 87 | 87.1 | 87.25 | 87.5 | 45 | 88.5 | 177 | 354 | 531 | 708 |

### Example 18

### ODT Pediatric Formulation

Orally disintegrating linaclotide tablets comprising components as shown in Table 38 and 39 may be prepared as described in Example 1. In addition, multiple dose compositions (e.g., 7-, 30-, 90-, 120-, and 180-dose compositions) may be prepared.

TABLE 38
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| PVP | 14 | 200 |
| Purified water, USP* | Q.S | Q.S. |
| Total | 70 | 1000 |

*Water is removed during the manufacturing process

TABLE 39
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| dihydrate | | | | | | | |
| Leucine | 0.0002 | 0.001 | 0.007 | 0.03 | 0.09 | 0.12 | 0.18 |
| PVP | 14 | 14 | 14 | 18 | 24 | 30 | 36 |
| Purified water, | Q.S | Q.S | Q.S | Q.S | Q.S | Q.S | Q.S |
| USP* | | | | | | | |
| Total (mg) | 70 | 70 | 70 | 90 | 90 | 110 | 120 |

*Water is removed during the manufacturing process

### Example 19

Orally disintegrating linaclotide tablets comprising components as shown in Table 40 and 41 may be prepared as described in Example 1. In addition, multiple dose compositions (e.g., 7-, 30-, 90-, 120-, and 180-dose compositions) may be prepared.

TABLE 40
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| PVA | 14 | 200 |
| Purified water, USP* | Q.S | Q.S. |
| Total | 70 | 1000 |

*Water is removed during the manufacturing process

TABLE 41
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| dihydrate | | | | | | | |
| Leucine | 0.0002 | 0.001 | 0.007 | 0.03 | 0.09 | 0.12 | 0.18 |
| PVA | 14 | 14 | 14 | 18 | 24 | 30 | 36 |
| Purified water, | Q.S | Q.S | Q.S | Q.S | Q.S | Q.S | Q.S |
| USP* | | | | | | | |
| Total (mg) | 70 | 70 | 70 | 90 | 90 | 110 | 120 |

*Water is removed during the manufacturing process

### Example 20

Orally disintegrating linaclotide tablets comprising components as shown in Table 42 and 43 may be prepared as described in Example 1. In addition, multiple dose compositions (e.g., 7-, 30-, 90-, 120-, and 180-dose compositions) may be prepared.

TABLE 42
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| HP-β-CD | 14 | 200 |
| Purified water, USP* | Q.S | Q.S. |
| Total | 70 | 1000 |

*Water is removed during the manufacturing process

TABLE 43
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| dihydrate | | | | | | | |
| Leucine | 0.0002 | 0.001 | 0.007 | 0.03 | 0.09 | 0.12 | 0.18 |
| HP-β-CD | 14 | 14 | 14 | 18 | 24 | 30 | 36 |
| Purified water, | Q.S | Q.S | Q.S | Q.S | Q.S | Q.S | Q.S |
| USP* | | | | | | | |
| Total (mg) | 70 | 70 | 70 | 90 | 90 | 110 | 120 |

*Water is removed during the manufacturing process

### Example 21

Orally disintegrating linaclotide tablets comprising components as shown in Table 44 and 45 may be prepared as described in Example 1. In addition, multiple dose compositions (e.g., 7-, 30-, 90-, 120-, and 180-dose compositions) may be prepared.

TABLE 44
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| Dextrin | 14 | 200 |
| Purified water, USP* | Q.S | Q.S. |
| Total | 70 | 1000 |

*Water is removed during the manufacturing process

TABLE 45
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| dihydrate | | | | | | | |
| Leucine | 0.0002 | 0.001 | 0.007 | 0.03 | 0.09 | 0.12 | 0.18 |
| dextrin | 14 | 14 | 14 | 18 | 24 | 30 | 36 |
| Purified water, | Q.S | Q.S | Q.S | Q.S | Q.S | Q.S | Q.S |
| USP* | | | | | | | |
| Total (mg) | 70 | 70 | 70 | 90 | 90 | 110 | 120 |

*Water is removed during the manufacturing process

### Example 22

Orally disintegrating linaclotide tablets comprising components as shown in Table 46 and 47 may be prepared as described in Example 1. In addition, multiple dose compositions (e.g., 7-, 30-, 90-, 120-, and 180-dose compositions) may be prepared.

TABLE 46
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| Carbopol | 14 | 200 |
| Purified water, USP* | Q.S | Q.S. |
| Total | 70 | 1000 |

*Water is removed during the manufacturing process

TABLE 47
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| dihydrate | | | | | | | |
| Leucine | 0.0002 | 0.001 | 0.007 | 0.03 | 0.09 | 0.12 | 0.18 |
| Carbopol | 14 | 14 | 14 | 18 | 24 | 30 | 36 |
| Purified water, | Q.S | Q.S | Q.S | Q.S | Q.S | Q.S | Q.S |
| USP* | | | | | | | |
| Total (mg) | 70 | 70 | 70 | 90 | 90 | 110 | 120 |

*Water is removed during the manufacturing process

### Example 23

Orally disintegrating linaclotide tablets comprising components as shown in Table 48 and 49 may be prepared as described in Example 1. In addition, multiple dose compositions (e.g., 7-, 30-, 90-, 120-, and 180-dose compositions) may be prepared.

TABLE 48
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| Gelatin | 14 | 200 |
| Purified water, USP* | Q.S | Q.S. |
| Total | 70 | 1000 |

*Water is removed during the manufacturing process

TABLE 49
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium chloride | 0.0006 | 0.003 | 0.021 | 0.09 | 0.27 | 0.36 | 0.54 |
| dihydrate | | | | | | | |
| Leucine | 0.0002 | 0.001 | 0.007 | 0.03 | 0.09 | 0.12 | 0.18 |
| Gelatin | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 24

Orally disintegrating linaclotide tablets comprising components as shown in Table 50 and 51 may be prepared as described in Example 1. In addition, multiple dose compositions (e.g., 7-, 30-, 90-, 120-, and 180-dose compositions) may be prepared.

TABLE 50
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| Hydropropylmethyl cellulose | 14 | 200 |
| Purified water, USP* | Q.S | Q.S. |
| Total | 70 | 1000 |

*Water is removed during the manufacturing process

TABLE 51
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium chloride | 0.0006 | 0.003 | 0.021 | 0.09 | 0.27 | 0.36 | 0.54 |
| dihydrate | | | | | | | |
| Leucine | 0.0002 | 0.001 | 0.007 | 0.03 | 0.09 | 0.12 | 0.18 |
| Hydropropyl | 14 | 14 | 14 | 18 | 24 | 30 | 36 |
| methyl cellulose | | | | | | | |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 25

Orally disintegrating linaclotide tablets comprising components as shown in Table 52 and 53 may be prepared as described in Example 1. In addition, multiple dose compositions (e.g., 7-, 30-, 90-, 120-, and 180-dose compositions) may be prepared.

TABLE 52
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| Hydropropyl cellulose | 14 | 200 |
| Purified water, USP* | Q.S | Q.S. |
| Total | 70 | 1000 |

*Water is removed during the manufacturing process

TABLE 53
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium chloride | 0.0006 | 0.003 | 0.021 | 0.09 | 0.27 | 0.36 | 0.54 |
| dihydrate | | | | | | | |
| Leucine | 0.0002 | 0.001 | 0.007 | 0.03 | 0.09 | 0.12 | 0.18 |
| Hydropropyl | 14 | 14 | 14 | 18 | 24 | 30 | 36 |
| cellulose | | | | | | | |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 26

Orally disintegrating linaclotide tablets comprising components as shown in Table 47 and 48 may be prepared as described in Example 1. In addition, multiple dose compositions (e.g., 7-, 30-, 90-, 120-, and 180-dose compositions) may be prepared.

TABLE 54
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| Hydropropyl cellulose | 14 | 200 |
| Purified water, USP* | Q.S | Q.S. |
| Total | 70 | 1000 |

*Water is removed during the manufacturing process

TABLE 55
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium chloride | 0.0006 | 0.003 | 0.021 | 0.09 | 0.27 | 0.36 | 0.54 |
| dihydrate | | | | | | | |
| Leucine | 0.0002 | 0.001 | 0.007 | 0.03 | 0.09 | 0.12 | 0.18 |
| Hydropropyl | 14 | 14 | 14 | 18 | 24 | 30 | 36 |
| cellulose | | | | | | | |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 27

Orally disintegrating linaclotide tablets comprising components as shown in Table 49 and 50 may be prepared as described in Example 1. In addition, multiple dose compositions (e.g., 7-, 30-, 90-, 120-, and 180-dose compositions) may be prepared.

TABLE 56
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| Methyl cellulose | 14 | 200 |
| Purified water, USP* | Q.S | Q.S. |
| Total | 70 | 1000 |

*Water is removed during the manufacturing process

TABLE 57
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium chloride | 0.0006 | 0.003 | 0.021 | 0.09 | 0.27 | 0.36 | 0.54 |
| dihydrate | | | | | | | |
| Leucine | 0.0002 | 0.001 | 0.007 | 0.03 | 0.09 | 0.12 | 0.18 |
| Methyl cellulose | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 28

Orally disintegrating linaclotide tablets comprising components as shown in Table 58 and 59 may be prepared as described in Example 1. In addition, multiple dose compositions (e.g., 7-, 30-, 90-, 120-, and 180-dose compositions) may be prepared.

TABLE 58
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride dihydrate | 0.003 | 0.04 |
| Leucine | 0.001 | 0.02 |
| Polyethylene oxide | 14 | 200 |
| Purified water, USP* | Q.S | Q.S. |
| Total | 70 | 1000 |

*Water is removed during the manufacturing process

TABLE 59
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium chloride | 0.0006 | 0.003 | 0.021 | 0.09 | 0.27 | 0.36 | 0.54 |
| dihydrate | | | | | | | |
| Leucine | 0.0002 | 0.001 | 0.007 | 0.03 | 0.09 | 0.12 | 0.18 |
| Polyethylene | 14 | 14 | 14 | 18 | 24 | 30 | 36 |
| oxide | | | | | | | |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 29

Orally disintegrating linaclotide tablets comprising components as shown in Table 60 and 61 may be prepared as described in Example 1. In addition, multiple dose compositions (e.g., 7-, 30-, 90-, 120-, and 180-dose compositions) may be prepared.

TABLE 60
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Sodium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 61
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Sodium chloride | 0.0006 | 0.003 | 0.021 | 0.09 | 0.27 | 0.36 | 0.54 |
| Leucine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 30

Orally disintegrating linaclotide tablets comprising components as shown in Table 62 and 63 may be prepared as described in Example 1. In addition, multiple dose compositions (e.g., 7-, 30-, 90-, 120-, and 180-dose compositions) may be prepared.

TABLE 62
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Zinc chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 63
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Zinc chloride | 0.0006 | 0.003 | 0.021 | 0.09 | 0.27 | 0.36 | 0.54 |
| Leucine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 31

Orally disintegrating linaclotide tablets comprising components as shown in Table 64 and 65 may be prepared as described in Example 1. In addition, multiple dose compositions (e.g., 7-, 30-, 90-, 120-, and 180-dose compositions) may be prepared.

TABLE 64
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Magnesium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 65
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Magnesium chloride | 0.0006 | 0.003 | 0.021 | 0.09 | 0.27 | 0.36 | 0.54 |
| Leucine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 32

Orally disintegrating linaclotide tablets comprising components as shown in Table 66 and 67 may be prepared as described in Example 1. In addition, multiple dose compositions (e.g., 7-, 30-, 90-, 120-, and 180-dose compositions) may be prepared.

TABLE 66
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Zinc chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 67
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Zinc chloride | 0.0006 | 0.003 | 0.021 | 0.09 | 0.27 | 0.36 | 0.54 |
| Leucine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 33

Orally disintegrating linaclotide tablets comprising components as shown in Table 68 and 69 may be prepared as described in Example 1. In addition, multiple dose compositions (e.g., 7-, 30-, 90-, 120-, and 180-dose compositions) may be prepared.

TABLE 68
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Aluminum chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 69
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Aluminum chloride | 0.0006 | 0.003 | 0.021 | 0.09 | 0.27 | 0.36 | 0.54 |
| Leucine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 34

Orally disintegrating linaclotide tablets comprising components as shown in Table 70 and 71 may be prepared as described in Example 1. In addition, multiple dose compositions (e.g., 7-, 30-, 90-, 120-, and 180-dose compositions) may be prepared.

TABLE 70
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Potassium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 71
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Potassium chloride | 0.0006 | 0.003 | 0.021 | 0.09 | 0.27 | 0.36 | 0.54 |
| Leucine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 35

Orally disintegrating linaclotide tablets comprising components as shown in Table 72 and 73 may be prepared as described in Example 1. In addition, multiple dose compositions (e.g., 7-, 30-, 90-, 120-, and 180-dose compositions) may be prepared.

TABLE 72
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Copper chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 73
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Copper chloride | 0.0006 | 0.003 | 0.021 | 0.09 | 0.27 | 0.36 | 0.54 |
| Leucine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 36

Orally disintegrating linaclotide tablets comprising components as shown in Table 74 and 75 may be prepared as described in Example 1. In addition, multiple dose compositions (e.g., 7-, 30-, 90-, 120-, and 180-dose compositions) may be prepared.

TABLE 74
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Isoleucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 75
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium chloride | 0.0006 | 0.003 | 0.021 | 0.09 | 0.27 | 0.36 | 0.54 |
| Isoleucine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 37

Orally disintegrating linaclotide tablets comprising components as shown in Table 76 and 77 may be prepared as described in Example 1. In addition, multiple dose compositions (e.g., 7-, 30-, 90-, 120-, and 180-dose compositions) may be prepared.

TABLE 76
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Glycine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 77
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium chloride | 0.0006 | 0.003 | 0.021 | 0.09 | 0.27 | 0.36 | 0.54 |
| Glycine | 0.0002 | 0.001 | 0.007 | 0.03 | 0.09 | 0.12 | 0.18 |
| Polyvinyl alcohol | 14 | 14 | 14 | 18 | 24 | 30 | 36 |
| Purified water, USP* | Q.S | Q.S | Q.S | Q.S | Q.S | Q.S | Q.S |
| Total (mg) | 70 | 70 | 70 | 90 | 90 | 110 | 120 |

*Water is removed during the manufacturing process

### Example 38

Orally disintegrating linaclotide tablets comprising components as shown in Table 78 and 79 may be prepared as described in Example 1. In addition, multiple dose compositions (e.g., 7-, 30-, 90-, 120-, and 180-dose compositions) may be prepared.

TABLE 78
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Histidine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 79
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Histidine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 39

Orally disintegrating linaclotide tablets comprising components as shown in Table 80 and 81 may be prepared as described in Example 1. In addition, multiple dose compositions (e.g., 7-, 30-, 90-, 120-, and 180-dose compositions) may be prepared.

TABLE 80
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Asparagine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 81
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Asparagine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 40

Orally disintegrating linaclotide tablets comprising components as shown in Table 82 and 83 may be prepared as described in Example 1. In addition, multiple dose compositions (e.g., 7-, 30-, 90-, 120-, and 180-dose compositions) may be prepared.

TABLE 82
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Alanine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 83
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Alanine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 41

Orally disintegrating linaclotide tablets comprising components as shown in Table 84 and 85 may be prepared as described in Example 1. In addition, multiple dose compositions (e.g., 7-, 30-, 90-, 120-, and 180-dose compositions) may be prepared.

TABLE 84
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Tyrosine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 85
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Tyrosine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 42

Orally disintegrating linaclotide tablets comprising components as shown in Table 86 and 87 may be prepared as described in Example 1. In addition, multiple dose compositions (e.g., 7-, 30-, 90-, 120-, and 180-dose compositions) may be prepared.

TABLE 86
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Cystine | 0.0001 | 0.02 |
| Polyvinyl alcohol | 14 | 200 |
| Purified water, USP* | Q.S | Q.S. |
| Total | 70 | 1000 |

*Water is removed during the manufacturing process

TABLE 87
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Cystine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 43

Orally disintegrating linaclotide tablets comprising components as shown in Table 88 and 89 may be prepared as described in Example 1. In addition, multiple dose compositions (e.g., 7-, 30-, 90-, 120-, and 180-dose compositions) may be prepared.

TABLE 88
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Proline | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 89
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Proline | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 44

Orally disintegrating linaclotide tablets comprising components as shown in Table 90 and 91 may be prepared as described in Example 1. In addition, multiple dose compositions (e.g., 7-, 30-, 90-, 120-, and 180-dose compositions) may be prepared.

TABLE 90
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Alanine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 91
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Alanine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 45

Orally disintegrating linaclotide tablets comprising components as shown in Table 92 and 93 may be prepared as described in Example 1. In addition, multiple dose compositions (e.g., 7-, 30-, 90-, 120-, and 180-dose compositions) may be prepared.

TABLE 92
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Lysine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 93
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Lysine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 46

Orally disintegrating linaclotide tablets comprising components as shown in Table 94 and 95 may be prepared as described in Example 1. In addition, multiple dose compositions (e.g., 7-, 30-, 90-, 120-, and 180-dose compositions) may be prepared.

TABLE 94
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Mannitol | 56 | 800 |
| Calcium chloride | 0.003 | 0.04 |
| Alanine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 95
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Mannitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Alanine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 47

Orally disintegrating linaclotide tablets comprising components as shown in Table 96 and 97 may be prepared as described in Example 1. In addition, multiple dose compositions (e.g., 7-, 30-, 90-, 120-, and 180-dose compositions) may be prepared.

TABLE 96
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Isomalt | 56 | 800 |
| Calcium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 97
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Isomalt | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Leucine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 48

Orally disintegrating linaclotide tablets comprising components as shown in Table 98 and 99 may be prepared as described in Example 1. In addition, multiple dose compositions (e.g., 7-, 30-, 90-, 120-, and 180-dose compositions) may be prepared.

TABLE 98
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Trehalose | 56 | 800 |
| Calcium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 99
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Trehalose | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Leucine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 49

Orally disintegrating linaclotide tablets comprising components as shown in Table 100 and 101 may be prepared as described in Example 1. In addition, multiple dose compositions (e.g., 7-, 30-, 90-, 120-, and 180-dose compositions) may be prepared.

TABLE 100
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Sorbitol | 56 | 800 |
| Calcium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 101
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Sorbitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Leucine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 50

Orally disintegrating linaclotide tablets comprising components as shown in Table 102 and 103 may be prepared as described in Example 1. In addition, multiple dose compositions (e.g., 7-, 30-, 90-, 120-, and 180-dose compositions) may be prepared.

TABLE 102
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Maltitol | 56 | 800 |
| Calcium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 103
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Maltitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Leucine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 51

Orally disintegrating linaclotide tablets comprising components as shown in Table 104 and 105 may be prepared as described in Example 1. In addition, multiple dose compositions (e.g., 7-, 30-, 90-, 120-, and 180-dose compositions) may be prepared.

TABLE 104
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Xylitol | 56 | 800 |
| Calcium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 105
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Xylitol | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Leucine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 52

Orally disintegrating linaclotide tablets comprising components as shown in Table 106 and 107 may be prepared as described in Example 1. In addition, multiple dose compositions (e.g., 7-, 30-, 90-, 120-, and 180-dose compositions) may be prepared.

TABLE 106
Linaclotide oral disintegrating tablet, 0.1 mcg/70 mg

| | Weight/tablet | Theoretical Weight |
|---|---|---|
| Components | (mg) | mg/g |
| Linaclotide | 0.0005 | 0.007 |
| Sucrose | 56 | 800 |
| Calcium chloride | 0.003 | 0.04 |
| Leucine | 0.001 | 0.02 |
| Polyvinyl alcohol | 14 | 200 |
| Purified water, USP* | Q.S | Q.S. |
| Total | 70 | 1000 |

*Water is removed during the manufacturing process

TABLE 107
Linaclotide oral disintegrating tablet of various strengths

| | Tablet composition of strength (mcg) | | | | | | |
|---|---|---|---|---|---|---|---|
| | 0.1 | 0.5 | 3.5 | 15 | 45 | 60 | 90 |
| | single | single | 7 dose | 30 dose | 90 dose | 120 dose | 180 dose |
| Components | dose | dose | comp | comp | comp | comp | comp |
| Linaclotide | 0.0001 | 0.0005 | 0.0035 | 0.015 | 0.045 | 0.06 | 0.09 |
| Sucrose | 56 | 56 | 56 | 72 | 65.6 | 79.5 | 73.2 |
| Calcium | 0.0006 | 0.003 | 0.021 | 0.09 | 0.27 | 0.36 | 0.54 |
| chloride | | | | | | | |
| Leucine | 0.0002 | 0.001 | 0.007 | 0.03 | 0.09 | 0.12 | 0.18 |
| Polyvinyl | 14 | 14 | 14 | 18 | 24 | 30 | 36 |
| alcohol | | | | | | | |
| Purified | Q.S | Q.S | Q.S | Q.S | Q.S | Q.S | Q.S |
| water, USP* | | | | | | | |
| Total (mg) | 70 | 70 | 70 | 90 | 90 | 110 | 120 |

*Water is removed during the manufacturing process

### Example 53

### Isolation and Preparation of Linaclotide Hydrolysis Product

The linaclotide hydrolysis product occurs as a transformation of Asn in the 7 position to Asp (the numbering of linaclotide starts with 1 at the N-terminal Cys). Its structure is depicted below:

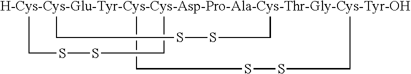

The linaclotide hydrolysis product has been independently synthesized for confirmation of identity using standard solid phase peptide synthesis techniques. The linaclotide hydrolysis product may also be prepared by other methods known in the art, e.g., by isolation from linaclotide preparations using chromatographic techniques or by recombinant expression of a nucleic acid encoding the linaclotide hydrolysis product (Cys Cys Glu Tyr Cys Cys Asp Pro Ala Cys Thr Gly Cys Tyr), optionally followed by oxidation of the cysteine residues to form the disulfide linkages.

### Example 54

### Isolation and Preparation of Linaclotide Formaldehyde Imine Product

The formaldehyde imine product occurs as the addition of an imine to the N-terminal Cys (Cys1) via a formaldehyde-mediated reaction. A proposed structure of the product is depicted below:

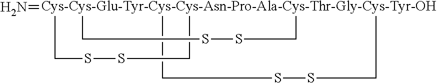

The linaclotide formaldehyde imine product has been independently synthesized for confirmation of identity by reacting linaclotide with formaldehyde (1:5 molar ratio) in absolute ethanol at room temperature for 4 days. The formaldehyde imine product may also be prepared by other methods known in the art, e.g., by isolation from linaclotide preparations using chromatographic techniques or by chemical peptide synthesis or recombinant expression of a nucleic acid encoding linaclotide followed by formylation as described herein or by other methods known in the art, optionally followed by oxidation of the cysteine residues to form the disulfide linkages.

### Example 55

### Isolation and Preparation of Linaclotide Oxidation Product

The linaclotide oxidation product has a molecular weight of 1542.8. The oxidation product most likely forms as the addition of a single oxygen atom to one of the six cysteinyl sulfurs in linaclotide. One potential structure of the product is depicted below, although one of skill in the art will recognize that the oxygen atom may be attached to any of the other five sulfurs:

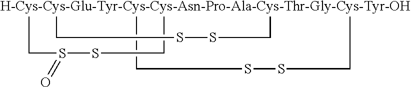

To support this identification, the linaclotide oxidation product has been produced by reacting linaclotide with hydrogen peroxide (3% aqueous) at room temperature or 40° C. for up to 24 hours. The resulting product is enriched in the oxidation product by 1-10%. The linaclotide oxidation product may also be prepared by other methods known in the art, e.g., by isolation from linaclotide preparations using chromatographic techniques or by chemical peptide synthesis or recombinant expression of a nucleic acid encoding linaclotide followed by oxidation of the cysteine residues to form the disulfide linkages followed by reacting linaclotide with hydrogen peroxide or similar oxidizing reagent to form the linaclotide oxidation product.

### Example 56

Orally disintegrating linaclotide tablets comprising components as shown in Table 108 were prepared in the manner described in Example 1. The stability, dissolution, and disintegration performance of the orally disintegrating linaclotide tablets (0.15 mg/90 mg, in aluminum pouch, with 2 grams desiccant) were evaluated as is illustrated in Table 109.

TABLE 108
Linaclotide ODT formulation, 150 mcg

| # | Ingredients | Wt. | | Wt % | Wt/tab |
|---|---|---|---|---|---|
| 1. | linaclotide | 11 | mg | 0.18 | 0.15 |
| 2. | Mannitol | 4.7 | g | 7.83 | 65.5 |
| 3. | Calcium chloride | 64 | mg | 1.06 | 0.9 |
| | dihydrate | | | | |
| 4. | Glycine | 15 | mg | 0.25 | 0.21 |
| 5. | Polyvinyl alcohol | 1.2 | g | 20 | 16.7 |
| | (Mw 30,000 to | | | | |
| | 70,000) | | | | |

| | | | | |
|---|---|---|---|---|
| 6. | Purified water | Q.S. | Q.S. | Q.S. |

| | | | | |
|---|---|---|---|---|
| Total weight | 6 | g | 100.03 | 83.5 |

TABLE 109
Stability of linaclotide Oral Disintegrating Tablet (0.15
mg/83.5 mg) in aluminum pouch with 2 g desiccant

| | Total | | | |
|---|---|---|---|---|
| | Linaclotide | | Dissolution % | |
| Condition | (mcg) | Total Deg % | 1 min | 5 min |
| Initial | 118.5 | 1.38 | 110 | 110 |
| 40/75, 1 month | 116.7 | 1.42 | 105 | 109 |
| 40/75, 2 month | 123 | 2.02 | 101 | 102 |
| 40/75, 3 month | 121.5 | 2.18 | 102 | 102 |
| 40/75, 6 month | 121.8 | 2.33 | 85.2 | 102 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

All patents, patent applications, publications, product descriptions, and protocols are cited throughout this application, the disclosures of which are incorporated herein by reference in their entireties for all purposes.

What is claimed is:

1. An orally disintegrating or dissolving pharmaceutical composition comprising linaclotide, or a pharmaceutically acceptable salt thereof.

2. The composition of claim 1, wherein the composition is an orally disintegrating or dissolving tablet.

3. The composition of claim 1, wherein the composition is an orally disintegrating or dissolving film.

4. The composition of any of claims 1-3, wherein the composition releases at least 70% of the linaclotide contained therein within 30 seconds of entering a use environment.

5. The composition of any of claims 1-3, wherein the composition releases at least 80% of the linaclotide contained therein within 30 seconds of entering a use environment.

6. The composition of any of claims 1-3, wherein the composition has a disintegration rate of less than 40 seconds.

7. The composition of any of claims 1-3, wherein the composition has a disintegration rate of less than 30 seconds.

8. The composition of any of claims 1-7, wherein the composition further comprises a stabilizing amount of a polymer, a stabilizing amount of a sterically hindered primary amine, or a stabilizing amount of a cation, or a combination or mixture thereof.

9. The composition of any of claims 1-8, wherein the composition comprises a stabilizing amount of a polymer.

10. The composition of any of claims 1-2 and 4-9, wherein the composition is an orally disintegrating tablet, and wherein the composition comprises 0.1 and 30% by weight of a polymer, relative to the total weight of the composition.

11. The composition of any of claims 1-2 and 4-9, wherein the composition is an orally disintegrating tablet, and wherein the composition comprises between 5 and 25% by weight of a polymer, relative to the total weight of the composition.

12. The composition of any of claims 1 and 3-9, wherein the composition is an orally disintegrating film, and wherein the composition comprises between 45 and 99% by weight of a polymer, relative to the total weight of the composition.

13. The composition of any of claims 1 and 3-9, wherein the composition is an orally disintegrating film, and wherein the composition comprises between 45 and 70% by weight of a polymer, relative to the total weight of the composition.

14. The composition of any of claims 1-13, wherein the polymer is polyvinyl pyrrolidone (PVP), polyvinyl alcohol (PVA), or a combination or mixture thereof.

15. The composition of claim 14, wherein the polymer is PVP.

16. The composition of claim 14, wherein the polymer is PVA.

17. The composition of any of claims 1-16, wherein the composition comprises a stabilizing amount of a sterically hindered primary amine.

18. The composition of any of claims 1-17, wherein the composition comprises a molar ratio of sterically hindered primary amine to linaclotide between 100:1 and 1:1.

19. The composition of any of claims 1-17, wherein the composition comprises a molar ratio of sterically hindered primary amine to linaclotide between 100:1 and 30:1.

20. The composition of any of claims 1-17, wherein the composition comprises a molar ratio of sterically hindered primary amine to linaclotide between 60:1 and 30:1.

21. The composition of any of claims 1-20, wherein the sterically hindered primary amine is an amino acid.

22. The composition of claim 21, wherein the amino acid is leucine, isoleucine, methionine, alanine, or a combination or mixture thereof.

23. The composition of claim 21, wherein the amino acid is leucine.

24. The composition of any of claims 1-23, wherein the composition comprises a stabilizing amount of a cation.

25. The composition of any of claims 1-24, wherein the composition comprises a molar ratio of cation to linaclotide between 100:1 and 1:1.

26. The composition of any of claims 1-24, wherein the composition comprises a molar ratio of cation to linaclotide between 100:1 and 40:1.

27. The composition of any of claims 1-24, wherein the composition comprises a molar ratio of cation to linaclotide between 100:1 and 60:1.

28. The composition of any of claims 1-27, wherein the cation is calcium, magnesium, manganese, zinc, potassium, sodium, or a mixture thereof.

29. The composition of any of claims 1-28, wherein the cation is a divalent metal cation.

30. The composition of claim 29, wherein the divalent metal cation is Ca²⁺, Mg²⁺, Mn²⁺, Zn²⁺, or a mixture thereof.

31. The composition of claim 29, wherein the divalent metal cation is Ca²⁺, Mg²⁺, Zn²⁺, or a mixture thereof.

32. The composition of claim 29, wherein the divalent metal cation is Ca²⁺.

33. The composition of claim 29, wherein the divalent metal cation is Mg²⁺.

34. The composition of claim 29, wherein the divalent metal cation is Zn²⁺.

35. The composition of any of claims 1-34, wherein the composition comprises a stabilizing amount of a polymer and stabilizing amount of a sterically hindered primary amine.

36. The composition of any of claims 1-35, wherein the composition comprises a stabilizing amount of a polymer and stabilizing amount of a cation.

37. The composition of any of claims 1-36, wherein the composition comprises a stabilizing amount of a sterically hindered primary amine and stabilizing amount of a cation.

38. The composition of any of claims 1-37, wherein the composition comprises a stabilizing amount of a polymer, a stabilizing amount of a sterically hindered primary amine, and stabilizing amount of a cation.

39. The composition of claim 1-38, wherein the composition comprises a stabilizing amount of a polymer selected from PVP and PVA and a stabilizing amount of an amino acid selected from leucine, isoleucine, alanine, and methionine.

40. The composition of claim 1-39, wherein the composition comprises a stabilizing amount of a polymer selected from PVP and PVA and a stabilizing amount of a cation selected from Ca²⁺, Mg²⁺, Zn²⁺, or a mixture thereof.

41. The composition of claim 1-40, wherein the composition comprises a stabilizing amount of an amino acid selected from leucine, isoleucine, alanine, and methionine, and a stabilizing amount of a cation selected from Ca²⁺, Mg²⁺, Zn²⁺, or a mixture thereof.

42. The composition of claim 1-41, wherein the composition comprises a stabilizing amount of a polymer selected from PVP and PVA, a stabilizing amount of an amino acid selected from leucine, isoleucine, alanine, and methionine, and a stabilizing amount of a cation selected from Ca²⁺, Mg²⁺, Zn²⁺, or a mixture thereof.

43. The composition of claim 42, wherein the composition is an orally disintegrating tablet, and wherein the composition comprises (i) between 0.1 and 30 wt. % by weight of a polymer selected from PVP and PVA, (ii) an amino acid selected from leucine, isoleucine, alanine, and methionine in a molar ratio of amino acid to linaclotide between 100:1 and 10:1, and (iii) a cation selected from Ca²⁺, Mg²⁺, Zn²⁺, or a mixture thereof, in a molar ratio of cation to linaclotide between 100:1 and 10:1.

44. The composition of claim 42, wherein the composition is an orally disintegrating tablet, and wherein the composition comprises (i) between 5 and 25 wt. % by weight of PVP, (ii) leucine in a molar ratio of leucine to linaclotide between 100:1 and 30:1, and (iii) Ca²⁺ in a molar ratio of Ca²⁺ to linaclotide between 100:1 and 60:1.

45. The composition of claim 42, wherein the composition is an orally disintegrating film, and wherein the composition comprises (i) between 45 and 99 wt. % by weight of a polymer selected from PVP and PVA, (ii) an amino acid selected from leucine, isoleucine, alanine, and methionine in a molar ratio of amino acid to linaclotide between 100:1 and 10:1, and (iii) a cation selected from Ca²⁺, Mg²⁺, Zn²⁺, or a mixture thereof, in a molar ratio of cation to linaclotide between 100:1 and 10:1.

46. The composition of claim 42, wherein the composition is an orally disintegrating film, and wherein the composition comprises (i) between 45 and 70 wt. % by weight of PVP, (ii) leucine in a molar ratio of leucine to linaclotide between 100:1 and 30:1, and (iii) Ca²⁺ in a molar ratio of Ca²⁺ to linaclotide between 100:1 and 60:1.

47. The composition of any of claims 1-46, wherein the composition further comprises a hydrolysis product having a structure of

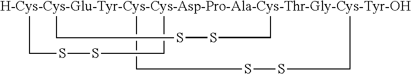

48. The composition of claim 47, wherein the composition comprises less than 5% by weight of the hydrolysis product.

49. The composition of claim 47, wherein the composition comprises from 0.05% to 5% by weight of the hydrolysis product.

50. The composition of claim 47, wherein the composition comprises from 0.05% to 2% by weight of the hydrolysis product.

51. The composition of any of claims 1-50, wherein the composition further comprises a formaldehyde imine product having a structure of:

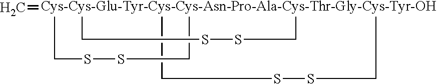

52. The composition of claim 51, wherein the composition comprises less than 5% by weight of the formaldehyde imine product.

53. The composition of claim 51, wherein the composition comprises from 0.05% to 5% by weight of the formaldehyde imine product.

54. The composition of claim 51, wherein the composition comprises from 0.05% to 2% by weight of the formaldehyde imine product.

55. The composition of any of claims 1-54, wherein the composition further comprises an oxidation product having a structure of:

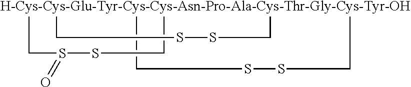

56. The composition of claim 55, wherein the composition comprises less than 5% by weight of the oxidation product.

57. The composition of claim 55, wherein the composition comprises from 0.05% to 5% by weight of the oxidation product.

58. The composition of claim 55, wherein the composition comprises from 0.05% to 2% by weight of the oxidation product.

59. The composition of any of claims 1-58, wherein the composition further comprises reduced form linaclotide.

60. The composition of claim 59, wherein the composition comprises less than 5% by weight of the reduced form linaclotide.

61. The composition of claim 59, wherein the composition comprises from 0.05% to 5% by weight of reduced form linaclotide.

62. The composition of claim 59, wherein the composition comprises from 0.05% to 2% by weight of reduced form linaclotide.

63. The composition of any of claims 1-62, wherein the composition further comprises scrambled form linaclotide.

64. The composition of claim 63, wherein the composition comprises less than 5% by weight of the scrambled form linaclotide.

65. The composition of claim 63, wherein the composition comprises from 0.05% to 5% by weight of scrambled form linaclotide.

66. The composition of claim 63, wherein the composition comprises from 0.05% to 2% by weight of scrambled form linaclotide.

67. The composition of any of claims 1-66, wherein the linaclotide is present in the composition in a concentration of 50 μg to 2 mg.

68. The composition of any of claims 1-67, wherein the linaclotide is present in the composition in a concentration of 75 μg, 150 μg, 300 μg, or 600 μg.

69. The composition of claim 68, wherein the linaclotide is present in the composition in a concentration of 75 μg.

70. The composition of claim 68, wherein the linaclotide is present in the composition in a concentration of 150 μg.

71. The composition of claim 68, wherein the linaclotide is present in the composition in a concentration of 300 μg.

72. The composition of claim 68, wherein the linaclotide is present in the composition in a concentration of 600 μg.

73. A method of treating a gastrointestinal disorder comprising administering to a patient in need thereof, a therapeutically effective amount of the composition of any of claims 1-72.

74. The method of claim 73, wherein the gastrointestinal disorder is selected from the group consisting of irritable bowel syndrome, constipation-predominant irritable bowel syndrome, chronic constipation, opioid induced constipation and dyspepsia.

75. A method of treating chronic constipation comprising administering to a patient in need thereof, a therapeutically effective amount of the composition of any of claims 1-72.

76. A method of treating irritable bowel syndrome comprising administering to a patient in need thereof, a therapeutically effective amount of the composition of any of claims 1-72.

77. A method of treating constipation-predominant irritable bowel syndrome comprising administering to a patient in need thereof, a therapeutically effective amount of the composition of any of claims 1-72.

78. A method of treating opioid induced constipation comprising administering to a patient in need thereof, a therapeutically effective amount of the composition of any of claims 1-72.

79. A method of treating dyspepsia comprising administering to a patient in need thereof, a therapeutically effective amount of the composition of any of claims 1-72.

80. A method of making the composition of any of claims 1-72, comprising:
i) preparing an aqueous solution comprises linaclotide, or a pharmaceutically acceptable salt thereof; and
ii) applying the aqueous solution to a pharmaceutically acceptable carrier.

81. A composition prepared by the method of claim 80.
